SEQUENCE LISTING (47 sequences)

SEQ ID NO: 1 - protein, 5 aa, synthetic construct
  REGION 1..5: note = Q-Tag 1
LLQGG

SEQ ID NO: 2 - protein, 4 aa, synthetic construct
  REGION 1..4: note = Q-Tag 2
LLQG

SEQ ID NO: 3 - protein, 6 aa, synthetic construct
  REGION 1..6: note = Q-Tag 3
LSLSQG

SEQ ID NO: 4 - protein, 8 aa, synthetic construct
  REGION 1..8: note = Q-Tag 4
GGGLLQGG

SEQ ID NO: 5 - protein, 5 aa, synthetic construct
  REGION 1..5: note = Q-Tag 5
GLLQG

SEQ ID NO: 6 - protein, 10 aa, synthetic construct
  REGION 1..10: note = Q-Tag 7
GSPLAQSHGG

SEQ ID NO: 7 - protein, 7 aa, synthetic construct
  REGION 1..7: note = Q-Tag 8
GLLQGGG

SEQ ID NO: 8 - protein, 6 aa, synthetic construct
  REGION 1..6: note = Q-Tag 9
GLLQGG

SEQ ID NO: 9 - protein, 4 aa, synthetic construct
  REGION 1..4: note = Q-Tag 10
GLLQ

SEQ ID NO: 10 - protein, 8 aa, synthetic construct
  REGION 1..8: note = Q-Tag 11
LLQLLQGA

SEQ ID NO: 11 - protein, 5 aa, synthetic construct
  REGION 1..5: note = Q-Tag 12
LLQGA

SEQ ID NO: 12 - protein, 7 aa, synthetic construct
  REGION 1..7: note = Q-Tag 13
LLQYQGA

SEQ ID NO: 13 - protein, 6 aa, synthetic construct
  REGION 1..6: note = Q-Tag 14
LLQGSG

SEQ ID NO: 14 - protein, 6 aa, synthetic construct
  REGION 1..6: note = Q-Tag 15
LLQYQG

SEQ ID NO: 15 - protein, 7 aa, synthetic construct
  REGION 1..7: note = Q-Tag 16
LLQLLQG

SEQ ID NO: 16 - protein, 5 aa, synthetic construct
  REGION 1..5: note = Q-Tag 17
SLLQG

SEQ ID NO: 17 - protein, 5 aa, synthetic construct
  REGION 1..5: note = Q-Tag 18
LLQLQ

SEQ ID NO: 18 - protein, 6 aa, synthetic construct
  REGION 1..6: note = Q-Tag 19
LLQLLQ

SEQ ID NO: 19 - protein, 5 aa, synthetic construct
  REGION 1..5: note = Q-Tag 20
LLQGR

SEQ ID NO: 20 - protein, 8 aa, synthetic construct
  REGION 1..8: note = Q-Tag 21
EEQYASTY

SEQ ID NO: 21 - protein, 8 aa, synthetic construct
  REGION 1..8: note = Q-Tag 22
EEQYQSTY

SEQ ID NO: 22 - protein, 8 aa, synthetic construct
  REGION 1..8: note = Q-Tag 23
EEQYNSTY

SEQ ID NO: 23 - protein, 6 aa, synthetic construct
  REGION 1..6: note = Q-Tag 24
EEQYQS

SEQ ID NO: 24 - protein, 7 aa, synthetic construct
  REGION 1..7: note = Q-Tag 25
EEQYQST

SEQ ID NO: 25 - protein, 7 aa, synthetic construct
  REGION 1..7: note = Q-Tag 26
EQYQSTY

SEQ ID NO: 26 - protein, 4 aa, synthetic construct
  REGION 1..4: note = Q-Tag 27
QYQS

SEQ ID NO: 27 - protein, 6 aa, synthetic construct
  REGION 1..6: note = Q-Tag 28
QYQSTY

SEQ ID NO: 28 - protein, 5 aa, synthetic construct
  REGION 1..5: note = Q-Tag 29
YRYRQ

SEQ ID NO: 29 - protein, 5 aa, synthetic construct
  REGION 1..5: note = Q-Tag 30
DYALQ

SEQ ID NO: 30 - protein, 7 aa, synthetic construct
  REGION 1..7: note = Q-Tag 31
FGLQRPY

SEQ ID NO: 31 - protein, 10 aa, synthetic construct
  REGION 1..10: note = Q-Tag 32
EQKLISEEDL

SEQ ID NO: 32 - protein, 335 aa, synthetic construct
  REGION 1..335: note = Streptomyces mobaraensis MTG Zedira
FRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 33 - protein, 335 aa, synthetic construct
  REGION 1..335: note = Streptomyces mobaraensis MTG
FRAPDSDERVTPPAEPLDRMPDPYRPSYGRAETIVNNYIRKWQQVYSHRDGRKQQMTEEQREWLSYGCVGVTWVNSGQYPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVTQGWP

SEQ ID NO: 34 - protein, 407 aa, synthetic construct [sequence omitted]

SEQ ID NO: 35 - protein, 395 aa, synthetic construct
  REGION 1..395: note = Streptoverticillium ladakanum MTG
MHRRIHAVGQARPPPTMARGKETKSYAETYRLTADDVANINALNESAPAASSAGPSFRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRSAPGTGLVDMSRDRNIPRSPTSPGEGFVNFDYGWFGAQTEADADKTVWTHGNHYHAPNGSLGCHACLTRASSATGSEGYSDFDRGEPYVVSPSPSPRMLEHRPRQGKAGLA

SEQ ID NO: 36 - protein, 446 aa, synthetic construct
  REGION 1..446: note = Polatuzumab heavy chain
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 218 aa, synthetic construct
  REGION 1..218: note = Polatuzumab light chain
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 450 aa, synthetic construct
  REGION 1..450: note = Trastuzumab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 214 aa, synthetic construct
  REGION 1..214: note = Trastuzumab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 447 aa, synthetic construct
  REGION 1..447: note = Enfortumab heavy chain
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 214 aa, synthetic construct
  REGION 1..214: note = Enfortumab light chain
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 447 aa, synthetic construct; identical to SEQ ID NO: 40
  REGION 1..447: note = Enfortumab heavy chain

SEQ ID NO: 43 - protein, 5 aa, synthetic construct
  REGION 1..5: note = Linker 1
RKAAR

SEQ ID NO: 44 - protein, 5 aa, synthetic construct
  REGION 1..5: note = Linker 2
RRKAY

SEQ ID NO: 45 - protein, 5 aa, synthetic construct
  REGION 1..5: note = Linker 4
ARKRA

SEQ ID NO: 46 - protein, 4 aa, synthetic construct
  REGION 1..4: note = Linker 5
RKAA

SEQ ID NO: 47 - protein, 4 aa, synthetic construct
  REGION 1..4: note = Linker 7
AAKR

(19) United States
(12) Patent Application Publication
(10) Pub. No.: US 2024/0398972 A1
(43) Pub. Date: Dec. 5, 2024

(54) METHODS FOR PRODUCING ANTIBODY-LINKER CONJUGATES

(71) Applicant: ARARIS BIOTECH AG, Au (CH)

(72) Inventors: Isabella ATTINGER-TOLLER, Au (CH); Romain BERTRAND, Au (CH); Ramona STARK, Au (CH); Dragan GRABULOVSKI, Au (CH); Philipp SPYCHER, Au (CH)

(21) Appl. No.: 18/639,584

(22) Filed: Apr. 18, 2024

Related U.S. Application Data
(63) Continuation of application No. PCT/EP2022/079787, filed on Oct. 25, 2022.

(30) Foreign Application Priority Data
Oct. 25, 2021 (EP) 21204588.4

(51) Int. Cl. A61K 47/68; C12N 9/10; C12P 21/00

(52) U.S. Cl. CPC A61K 47/6889; A61K 47/68031; A61K 47/68033; A61K 47/68037; A61K 47/6849; A61K 47/6855; C12N 9/1044; C12P 21/005; C12Y 203/02013

(57) ABSTRACT

The present invention relates to a method for generating an antibody-payload conjugate by means of a transglutaminase. The method comprises a step of conjugating a linker comprising the structure (shown in N→C direction) (Sp₁)-K-(Sp₂)-B-(Sp₃) or (Sp₁)-B-(Sp₂)-K-(Sp₃) to a Gln residue comprised in an antibody, wherein (Sp₁) is a chemical spacer or is absent; (Sp₂) is a chemical spacer or is absent; (Sp₃) is a chemical spacer or is absent; K is lysine or a lysine derivative or a lysine mimetic; B is a linking moiety or a payload; wherein the linker is conjugated to the Gln residue comprised in the antibody via a primary amine comprised in the side chain of the lysine residue, the lysine derivative or the lysine mimetic; and wherein the antibody is contacted with less than 80 molar equivalents of the linker. Further, the invention relates to antibody-linker conjugates, antibody-drug conjugates and linker constructs comprising a lysine residue.

### RELATED APPLICATIONS

This application is a continuation of International Patent Application No. PCT/EP2022/079787, filed Oct. 25, 2022, which claims priority to European Patent Application No. 21204588.4, filed Oct. 25, 2021, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Apr. 18, 2024, is named 751861_ARA9-006PCCON_ST26.xml and is 62,756 bytes in size.

The present invention relates to methods for generating an antibody-linker conjugate by means of a transglutaminase. The invention further provides antibody-linker conjugates and pharmaceutical compositions comprising the antibody-linker conjugates of the invention and uses thereof.

Antibody-drug conjugates (ADCs) are typically composed of an antibody and a small molecule drug conjugated to the antibody via a chemical linker. After decades of preclinical and clinical studies, a series of ADCs have been approved for treating specific tumor types such as brentuximab vedotin (Adcetris®) for relapsed Hodgkin's lymphoma and systemic anaplastic large cell lymphoma, gemtuzumab ozogamicin (Mylotarg®) for acute myeloid leukemia, ado-trastuzumab emtansine (Kadcyla®) for HER2-positive metastatic breast cancer, inotuzumab ozogamicin (Besponsa®) and recently polatuzumab vedotin-piiq (Polivy®) for B cell malignancies. Most recently, enfortumab vedotin (Padcev®), trastuzumab deruxtecan (Enhertu®), sacituzumab govitecan (Trodelvy®) and belantamab mafadotin (Blenrep®) have received market approval. For a review on ADCs see for example (Zhao P. et al., 2020, Acta Pharmaceutica Sinica B, 10, 1589-1600). While many ADCs have shown impressive anti-cancer activity, many patients do not respond to these treatments, experience severe side-effects before signs of efficacy or experience a relapse after a certain period of time, so there is still a large medical need for novel ADC formats which have favorable drug-like properties, can be produced in sufficient quantity and quality at reasonable costs to support drug development, and which are suitable as therapeutics.

A key step in the preparation of an ADC is the covalent conjugation step of a payload to the antibody. Most ADCs in current clinical development were made by conjugation to endogenous lysine or cysteine residues of the antibody, carefully controlling the average degree of modification to yield an average drug-to-antibody ratio (DAR) in the range of 3.5-4.0. This ratio was historically selected on the basis of (a) minimizing the amount of nonconjugated antibody and (b) avoiding species in the mixture with very high DAR, which may be problematic in manufacturing and formulation because of higher hydrophobicity and lower solubility (Lambert J M and Berkenbilt A., 2018, Annu. Rev. Med. 69, 191-207) and typically results in poor pharmacokinetic properties (Lyon R P, et al., 2015, Nat Biotechnol, 33, 733-735). Recently, a variety of genetic, chemical, and enzymatic methods have been developed for site-specific conjugation, which can enable DARs of 2 (or 4) while avoiding under- or overmodification of the antibody. An overview of these methodologies are given in Yamada et al. (reviewed in Kei Yamada and Yuji Ito, 2019, ChemBioChem, 20, 2729-2739).

Enzymatic conjugation has shown great interest since these conjugation reactions are typically fast, site-specific and can be done under physiological conditions. Among the available enzymes, microbial transglutaminase (MTG) from the species Streptomyces mobaraensis has found increasing interest as an attractive alternative to conventional chemical protein conjugation of functional moieties including antibodies. The MTG catalyzes under physiological conditions a transamidation reaction between a ‘reactive’ glutamine of a protein or peptide and a ‘reactive’ lysine residue of a protein or peptide, whereas the latter can also be a simple, low molecular weight primary amine such as a 5-aminopentyl group (Jeger S. et al., 2010, Angew. Chem. Int. Ed., 49, 9995-9997).

Jeger et al. described that conjugation of an antibody using transglutaminase as an enzyme happens at the Q295 residue, however, conjugation was only possible upon removal of the glycan moiety at the asparagine residue 297 (N297) with PNGase F, while glycosylated antibodies could not be conjugated efficiently (conjugation efficiency below 20%) (Jeger S. et al., 2010, Angew. Chem. Int. Ed., 49, 9995-9997; Mindt T. et al. 2008, Bioconj Chem, 9, 271-278).

Other approaches to generate ADCs by means of an MTG are based on the use of aglycosylated antibodies, wherein residue N297 is substituted with an amino acid residue that cannot be subject to glycosylation. However, substitution of N297 against another amino acid may lead to unwanted effects, as it may affect the overall stability of the entire Fc domain (Subedi G P and Barb A W., 2015, Structure, 23, 1573-1583), and the efficacy of the entire conjugate. As a consequence that can lead to increased antibody aggregation and a decreased solubility that particularly becomes important for hydrophobic payloads. Further, the glycan that is present at N297 has important immunomodulatory effects, as it triggers effector functions such as antibody dependent cellular cytotoxicity (ADCC) and the like. These immunomodulatory effects would get lost upon deglycosylation or any of the other approaches discussed above to obtain an aglycosylated antibody. Further, any sequence modification of an established antibody can also lead to regulatory problems, which is problematic because often times an accepted and clinically validated antibody is used as a starting point for ADC conjugation.

Recently, Spycher et al. disclosed a transglutaminase-based conjugation approach which does not require prior deglycosylation of the antibody for payload conjugation (Spycher et al., WO 2019/057772). The possibility to conjugate native, glycosylated antibodies offers significant advantages under manufacturing aspects: an enzymatic deglycosylation step is undesired under good manufacturing process (GMP) aspects, because it has to be made sure that the both the deglycosylation enzyme (e.g., PNGase F) as well as the cleaved glycan are removed from the reaction mixture. Furthermore, no genetic engineering of the antibody for payload attachment is necessary, so that sequence insertions which may increase immunogenicity and decrease the overall stability of the antibody can be avoided.

In view of the foregoing, there is still a need in the art for improved methods for generating ADCs with a high conjugation efficiency.

### SUMMARY OF THE INVENTION

The present invention is characterized in the herein provided embodiments and claims. In particular, the present invention relates, inter alia, to the following embodiments:
1. A method for producing an antibody-linker conjugate by means of a transglutaminase, the method comprising a step of conjugating a linker comprising the structure (shown in N→C direction)

(Sp₁)-K-(Sp₂)-B-(Sp₃) or(Sp₁)-B-(Sp₂)-K-(Sp₃)
to a Gln residue comprised in an antibody, wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   B is a linking moiety or a payload;
wherein the linker is conjugated to the Gln residue comprised in the antibody via a primary amine comprised in the side chain of the lysine residue, the lysine derivative or the lysine mimetic; and
wherein the antibody is contacted with less than 80 molar equivalents of the linker.
2. The method according to embodiment 1, wherein the antibody is contacted with molar equivalents or less than 20 molar equivalents of the linker.
3. The method according to embodiment 1 or 2, wherein the antibody is contacted with 2-20 molar equivalents of the linker.
4. The method according to any one of embodiments 1-3, wherein the antibody is added to the conjugation reaction at a concentration ranging from 1-50 mg/mL.
5. The method according to any one of embodiments 1-4, wherein the transglutaminase is added to the conjugation reaction at a concentration ranging from 1-20 U/mg antibody.
6. The method according to any one of embodiments 1-5, wherein conjugation of the linker to the antibody is achieved at a pH ranging from 6 to 8.5.
7. The method according to any one of embodiments 1-6, wherein the chemical spacers (Sp₁), (Sp₂) and (Sp₃) each independently comprise between 0 and 12 amino acid residues.
8. The method according to any one of embodiments 1-7, wherein the linker comprises not more than 25, 20, 15, 14, 13, 12, 11, 10, 9, 8, 7, 6, 5, 4 amino acid residues.
9. The method according to any one of embodiments 1-8, wherein the net charge of the linker is neutral or positive.
10. The method according to any one of embodiments 1-9, wherein the linker comprises no negatively-charged amino acid residues.
11. The method according to any one of embodiments 1-10, wherein B is a linking moiety.
12. The method according to embodiment 11, wherein the linking moiety B comprises
   a bioorthogonal marker group, or
   a non-bio-orthogonal entity for crosslinking.
13. The method according to embodiment 12, wherein the bioorthogonal marker group or the non-bio-orthogonal entity for crosslinking consists of or comprises at least one molecule or moiety selected from the group consisting of:
   —N—N≡N, or —N₃;
   Lys(N₃);
   a tetrazine;
   an alkyne;
   a strained cyclooctyne;
   BCN;
   a strained alkene;
   a photoreactive group;
   an aldehyde;
   an acyltrifluoroborate;
   a protein degradation agent (‘PROTAC’);
   a cyclopentadiene/spirolocyclopentadiene;
   a thio-selective electrophile;
   —SH; and
   cysteine.
14. The method according to any one of embodiments 11-13, the method comprising a further step of conjugating one or more payloads to the linking moiety B.
15. The method according to embodiment 14, wherein the one or more payload is conjugated to the linking moiety B via a click-reaction.
16. The method according to anyone of embodiments 1-10, wherein B is a payload.
17. The method according to any one of embodiments 14 to 16, wherein the payload comprises at least one of:
   a toxin;
   a cytokine;
   a growth factor;
   a radionuclide;
   a hormone;
   an anti-viral agent;
   an anti-bacterial agent;
   a fluorescent dye;
   an immunoregulatory/immunostimulatory agent;
   a half-life increasing moiety;
   a solubility increasing moiety;
   a polymer-toxin conjugate;
   a nucleic acid;
   a biotin or streptavidin moiety;
   a vitamin;
   a protein degradation agent (‘PROTAC’);
   a target binding moiety; and/or
   an anti-inflammatory agent.
18. The method according to embodiment 17, wherein the toxin is at least one selected from the group consisting of
   a pyrrolobenzodiazepine (e.g., PBD);
   an auristatin (e.g., MMAE, MMAF);
   a maytansinoid (e.g., maytansine, DM1, DM4, DM21);
   a duocarmycin;
   a nicotinamide phosphoribosyltransferase (NAMPT) inhibitor;
   a tubulysin;
   an enediyne (e.g., calicheamicin);
   an anthracycline derivative (PNU) (e.g., doxorubicin);
   a pyrrole-based kinesin spindle protein (KSP) inhibitor;
   a cryptophycin;
   a drug efflux pump inhibitor;
   a sandramycin;
   an amanitin (e.g., α-amanitin); and
   a camptothecin (e.g., exatecans, deruxtecans).
19. The method according to any one of embodiments 1 to 18, wherein the chemical spacer (Sp₂) comprises a self-immolative moiety.
20. The method according to embodiment 19, wherein the self-immolative moiety is directly attached to the payload B.
21. The method according to embodiment 19 or 20, wherein the self-immolative moiety comprises a p-aminobenzyl carbamoyl (PABC) moiety or a self-immolative amino methylene spacer.
22. The method according to any one of embodiments 1 to 21, wherein the antibody is an IgG antibody, in particular an IgG1 antibody.
23. The method according to embodiment 22, wherein the Gln residue to which the linker is conjugated is comprised in an Fc domain of the antibody, in particular wherein the Gln residue to which the linker is conjugated is Gln residue Q295 (EU numbering) of the C_{H}2 domain of an IgG antibody.
24. The method according to embodiment 22, wherein the Gln residue to which the linker is conjugated has been introduced into the heavy or light chain of the antibody by molecular engineering.
25. The method according to embodiment 24, wherein the Gln residue that has been introduced into the heavy or light chain of the antibody by molecular engineering is N297Q (EU numbering) of the C_{H}2 domain of an aglycosylated IgG antibody.
26. The method according to embodiment 24, wherein the Gln residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.
27. The method according to embodiment 26, wherein the peptide comprising the Gln residue has been fused to the C-terminal end of the heavy chain of the antibody.
28. The method according to any one of embodiments 22 to 24 or 26 to 27, wherein the IgG antibody is a glycosylated IgG antibody, in particular wherein the IgG antibody is glycosylated at residue N297 (EU numbering) of the C_{H}2 domain.
29. The method according to any one of embodiments 1 to 28, wherein the antibody is selected from the group consisting of: Brentuximab, Trastuzumab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Sacituzumab, Belantamab, Polatuzumab and Enfortumab.
30. The method according to any one of embodiments 1 to 29, wherein the linker is conjugated to the γ-carboxamide group of the Gln residue comprised in the antibody.
31. The method according to any one of embodiments 1 to 30, wherein the linker is suitable for conjugation to a glycosylated antibody with a conjugation efficiency of at least 20%, 30%, 40%, 50%, 60%, 70%, 75%, 80%, 85%, 90% or 95%.
32. The method according to any one of embodiments 1 to 31, wherein the transglutaminase is a microbial transglutaminase, preferably derived from a Streptomyces species, in particular Streptomyces mobaraensis.
33. An antibody-linker conjugate which has been produced with a method according to any one of embodiments 1 to 32.
34. A pharmaceutical composition comprising the antibody-linker conjugate according to embodiment 33.
35. The pharmaceutical composition according to embodiment 34 comprising at least one additional therapeutically active agent.
36. The antibody-linker conjugate according to embodiment 33 or the pharmaceutical composition according to embodiment 34 or 35 for use in therapy and/or diagnostics, in particular wherein the antibody-linker conjugate comprises at least one payload.
37. The antibody-linker conjugate according to embodiment 33 or the pharmaceutical composition according to embodiment 34 or 35 for use in the treatment of a patient
   suffering from,
   being at risk of developing, and/or
   being diagnosed for
      a neoplastic disease, a neurological disease, an autoimmune disease, an inflammatory disease or an infectious disease, in particular wherein the antibody-linker conjugate comprises at least one toxin.
38. The antibody-linker conjugate or the pharmaceutical composition for use according to embodiment 37, wherein the antibody-linker conjugate comprises Polatuzumab and wherein the neoplastic disease is a B-cell associated cancer.
39. The antibody-linker conjugate or the pharmaceutical composition for use according to embodiment 38, wherein the B-cell associated cancer is non-Hodgkin lymphoma, in particular wherein the B-cell associated cancer is diffuse large B-cell lymphoma.
40. The antibody-linker conjugate or the pharmaceutical composition for use according to embodiment 37, wherein the antibody-linker conjugate comprises Trastuzumab and wherein the neoplastic disease is a HER2-positive cancer, in particular HER2-positive breast, gastric, ovarian or lung cancer.
41. The antibody-linker conjugate or the pharmaceutical composition for use according to embodiment 37, wherein the antibody-linker conjugate comprises Enfortumab or an Enfortumab variant and wherein the neoplastic disease is a Nectin-4 positive cancer, in particular Nectin-4 positive pancreatic cancer, lung cancer, bladder cancer or breast cancer.

Accordingly, in one embodiment, the invention relates to a method for producing an antibody-linker conjugate by means of a transglutaminase, the method comprising a step of conjugating a linker comprising the structure (shown in N→C direction)

(Sp₁)-K-(Sp₂)-B-(Sp₃) or(Sp₁)-B-(Sp₂)-K-(Sp₃)
to a Gln residue comprised in an antibody, wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   B is a linking moiety or a payload;
wherein the linker is conjugated to the Gln residue comprised in the antibody via a primary amine comprised in the side chain of the lysine residue, the lysine derivative or the lysine mimetic; and
wherein the antibody is contacted with less than 80 molar equivalents of the linker.

That is, the invention is based, at least in part, on the surprising finding that lysine-based linkers can be conjugated to glycosylated antibodies with exceptionally high efficiencies under optimized reaction conditions. In particular, it has been shown by the inventors that an optimized linker-to-antibody ratio results in surprisingly high conjugation efficiencies.

In WO 2019/057772, it has been demonstrated for the first time that lysine-based peptide linkers can be conjugated to glycosylated antibodies. To achieve efficient conjugation, it was believed in the art that glycosylated antibodies have to be contacted with large excess of linker. Consequently, glycosylated antibodies have been contacted with 80 molar equivalents of a lysine-based linker to achieve conjugation in WO 2019/057772. Contrary to what was believed, the inventors surprisingly found that lowering the linker-to-antibody ratio results in significantly more efficient conjugation of glycosylated antibodies. As a result, quantitative conjugation of various linkers comprising bulky payloads has been achieved in a single step.

In particular, it has been demonstrated that contacting the antibody with less than 80 molar equivalents of a linker results in improved conjugation efficiencies. In a preferred embodiment, the antibody is contacted with less than 70 molar equivalents of a linker. In a more preferred embodiment, the antibody is contacted with less than 60 molar equivalents of a linker. In an even more preferred embodiment, the antibody is contacted with less than 50 molar equivalents of a linker. In an even more preferred embodiment, the antibody is contacted with less than 40 molar equivalents of a linker. In an even more preferred embodiment, the antibody is contacted with less than 30 molar equivalents of a linker. In an even more preferred embodiment, the antibody is contacted with less than 20 molar equivalents of a linker. In an even more preferred embodiment, the antibody is contacted with less than 15 molar equivalents of a linker. In a most preferred embodiment, the antibody is contacted with less than 10 molar equivalents of a linker.

Alternatively, the antibody may be contacted with 2-80 molar equivalents of linker, preferably 2-70 molar equivalents of linker, more preferably 2-60 molar equivalents of linker, even more preferably 2-50 molar equivalents of linker, even more preferably 2-40 molar equivalents of linker, even more preferably 2-30 molar equivalents of linker, even more preferably 2-25 molar equivalents of linker, even more preferably 2 to 20 molar equivalents of linker, even more preferably 2-15 molar equivalents of linker, even more preferably 2-10 molar equivalents of linker, most preferably 2-8 molar equivalents of linker.

Alternatively, the antibody may be contacted with 2.5-80 molar equivalents of linker, preferably 2.5-70 molar equivalents of linker, more preferably 2.5-60 molar equivalents of linker, even more preferably 2.5-50 molar equivalents of linker, even more preferably 2.5-40 molar equivalents of linker, even more preferably 2.5-30 molar equivalents of linker, even more preferably 2.5-25 molar equivalents of linker, even more preferably 2.5-20 molar equivalents of linker, even more preferably 2.5-15 molar equivalents of linker, even more preferably 2.5-10 molar equivalents of linker, most preferably 2.5-8 molar equivalents of linker.

The antibody may be added to the conjugation reaction in any concentration. However, it is preferred that the antibody is added to the conjugation reaction at a concertation ranging from 0.1-50 mg/ml. That is, in a particular embodiment, the invention relates to the method according to the invention, wherein the antibody is added to the conjugation reaction at a concentration ranging from 0.1-50 mg/mL, preferably from 1-50 mg/ml, more preferably from 1-25 mg/mL, more preferably from 2.5-20 mg/mL, even more preferably from 5-20 mg/mL, most preferably from 5-17 mg/mL.

The method according to the invention is preferably carried out at a pH ranging from 6 to 9. Thus, in a preferred embodiment, the invention relates to a method according to the invention, wherein the conjugation of the linker to the antibody is achieved at a pH ranging from 6 to 8.5, more preferably at a pH ranging from 6.5 to 8, even more preferably at a pH ranging from 7 to 8. In a most preferred embodiment, the invention relates to a method according to the invention, wherein the conjugation of the linker to the antibody is achieved at pH 7.6.

The method of the invention may be carried out in any buffer that is suitable for the conjugation of the payload to the linker. Buffers that are suitable for the method of the invention include, without limitation, Tris, MOPS, HEPES, PBS or BisTris buffer. The concentration of the buffer depends, amongst others, on the concentration of the antibody and/or the linker and may range from 10-1000 mM, 10-500 mM, 10-400 mM, 10 to 250 mM, 10 to 150 mM or 10 to 100 mM. Further, the buffer may comprise any salt concentration that is suitable for carrying out the method of the invention. For example, the buffer used in the method of the invention may have a salt concentration 150 mM, ≤140 mM, ≤130 mM, 120 mM, ≤110 mM, ≤100 mM, ≤90 mM, ≤80 mM, ≤70 mM, ≤60M, ≤50 mM, ≤40 mM, 30 mM, ≤20 mM or 10 mM or no salts. In a particular embodiment, the method of the invention is carried out in 50 mM Tris (pH 7.6), preferably without salts.

It has to be noted that the optimal reaction conditions (e.g. pH, buffer, salt concentration) may vary between payloads and to some degree depend on the physicochemical properties of the linkers and/or payloads. However, no undue experimentation is required by the skilled person to identify reaction conditions that are suitable for carrying out the method of the invention.

Transglutaminase may be added to the conjugation reaction at any concentration that allows efficient conjugation of an antibody with a linker. In certain embodiments, the concentration of transglutaminase in a conjugation reaction may depend on the amount of antibody used in the same reaction. For example, a transglutaminase may be added to the conjugation reaction at a concentration of less than 100 U/mg antibody, 90 U/mg antibody, 80 U/mg antibody, 70 U/mg antibody, 60 U/mg antibody, 50 U/mg antibody, 40 U/mg antibody, 30 U/mg antibody, U/mg antibody 10 U/mg antibody or 6 U/mg antibody. In certain embodiments a transglutaminase may be added to the conjugation reaction at a concentration of 1, 3, 5 or 6 U/mg antibody.

That is, in certain embodiments, a transglutaminase may be added to the conjugation reaction at a concentration ranging from 1-20 U/mg antibody, preferably 1-10 U/mg antibody, more preferably 1-7.5 U/mg antibody, even more preferably 2-6 U/mg antibody, even more preferably 2-4 U/mg antibody, most preferably 3 U/mg antibody.

The method according to the invention is preferably catalyzed by a microbial transglutaminase. However, it is to be noted that an equivalent reaction may be carried out by an enzyme comprising transglutaminase activity that is of a non-microbial origin. Accordingly, the antibody-linker conjugates according to the invention may be generated with an enzyme comprising transglutaminase activity that is of a non-microbial origin.

It is to be understood that the application encompasses any combination of the above-disclosed linker, antibody, transglutaminase and/or buffer concentrations.

In a preferred embodiment, the invention relates to a method for producing an antibody-linker conjugate by means of a transglutaminase, the method comprising a step of conjugating a linker comprising the structure (shown in N→C direction)

(Sp₁)-K-(Sp₂)-B-(Sp₃) or(Sp₁)-B-(Sp₂)-K-(Sp₃)
to a Gln residue comprised in an antibody, wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   B is a linking moiety or a payload;
wherein the linker is conjugated to the Gln residue comprised in the antibody via a primary amine comprised in the side chain of the lysine residue, the lysine derivative or the lysine mimetic; and
wherein the antibody is contacted with 2-70 molar equivalents of the linker; and/or wherein the antibody is added to the conjugation reaction at a concentration ranging from 0.1-50 mg/mL; and, optionally, wherein the transglutaminase is added to the conjugation reaction at a concentration ranging from 1-20 U/mg antibody.

In a more preferred embodiment, the invention relates to a method for producing an antibody-linker conjugate by means of a transglutaminase, the method comprising a step of conjugating a linker comprising the structure (shown in N→C direction)

(Sp₁)-K-(Sp₂)-B-(Sp₃) or(Sp₁)-B-(Sp₂)-K-(Sp₃)
to a Gln residue comprised in an antibody, wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   is a linking moiety or a payload;
wherein the linker is conjugated to the Gln residue comprised in the antibody via a primary amine comprised in the side chain of the lysine residue, the lysine derivative or the lysine mimetic; and
wherein the antibody is contacted with 2-50 molar equivalents of the linker; and/or wherein the antibody is added to the conjugation reaction at a concentration ranging from 1-50 mg/mL; and, optionally, wherein the transglutaminase is added to the conjugation reaction at a concentration ranging from 1-20 U/mg antibody.

In an even more preferred embodiment, the invention relates to a method for producing an antibody-linker conjugate by means of a transglutaminase, the method comprising a step of conjugating a linker comprising the structure (shown in N→C direction)

(Sp₁)-K-(Sp₂)-B-(Sp₃) or(Sp₁)-B-(Sp₂)-K-(Sp₃)
to a Gln residue comprised in an antibody, wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   B is a linking moiety or a payload;
wherein the linker is conjugated to the Gln residue comprised in the antibody via a primary amine comprised in the side chain of the lysine residue, the lysine derivative or the lysine mimetic; and
wherein the antibody is contacted with 2-25 molar equivalents of the linker; and/or wherein the antibody is added to the conjugation reaction at a concentration ranging from 2.5-25 mg/mL; and, optionally, wherein the transglutaminase is added to the conjugation reaction at a concentration ranging from 1-15 U/mg antibody.

In an even more preferred embodiment, the invention relates to a method for producing an antibody-linker conjugate by means of a transglutaminase, the method comprising a step of conjugating a linker comprising the structure (shown in N→C direction)

(Sp₁)-K-(Sp₂)-B-(Sp₃) or(Sp₁)-B-(Sp₂)-K-(Sp₃)
to a Gln residue comprised in an antibody, wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   B is a linking moiety or a payload;
wherein the linker is conjugated to the Gln residue comprised in the antibody via a primary amine comprised in the side chain of the lysine residue, the lysine derivative or the lysine mimetic; and
   wherein the antibody is contacted with 2-20 molar equivalents of the linker; and/or wherein the antibody is added to the conjugation reaction at a concentration ranging from 2.5-20 mg/mL; and, optionally, wherein the transglutaminase is added to the conjugation reaction at a concentration ranging from 1-15 U/mg antibody.

In an even more preferred embodiment, the invention relates to a method for producing an antibody-linker conjugate by means of a transglutaminase, the method comprising a step of conjugating a linker comprising the structure (shown in N→C direction)

(Sp₁)-K-(Sp₂)-B-(Sp₃) or(Sp₁)-B-(Sp₂)-K-(Sp₃)
to a Gln residue comprised in an antibody, wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   B is a linking moiety or a payload;
wherein the linker is conjugated to the Gln residue comprised in the antibody via a primary amine comprised in the side chain of the lysine residue, the lysine derivative or the lysine mimetic; and
   wherein the antibody is contacted with 2.5-15 molar equivalents of the linker; and/or wherein the antibody is added to the conjugation reaction at a concentration ranging from 2.5-20 mg/mL; and, optionally, wherein the transglutaminase is added to the conjugation reaction at a concentration ranging from 1-10 U/mg antibody.

In an even more preferred embodiment, the invention relates to a method for producing an antibody-linker conjugate by means of a transglutaminase, the method comprising a step of conjugating a linker comprising the structure (shown in N→C direction)

(Sp₁)-K-(Sp₂)-B-(Sp₃) or(Sp₁)-B-(Sp₂)-K-(Sp₃)
to a Gln residue comprised in an antibody, wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   B is a linking moiety or a payload;
wherein the linker is conjugated to the Gln residue comprised in the antibody via a primary amine comprised in the side chain of the lysine residue, the lysine derivative or the lysine mimetic; and
   wherein the antibody is contacted with 2.5-10 molar equivalents of the linker; and/or wherein the antibody is added to the conjugation reaction at a concentration ranging from 5-20 mg/mL; and, optionally, wherein the transglutaminase is added to the conjugation reaction at a concentration ranging from 2-10 U/mg antibody.

In a most preferred embodiment, the invention relates to a method for producing an antibody-linker conjugate by means of a transglutaminase, the method comprising a step of conjugating a linker comprising the structure (shown in N→C direction)

(Sp₁)-K-(Sp₂)-B-(Sp₃) or(Sp₁)-B-(Sp₂)-K-(Sp₃)
to a Gln residue comprised in an antibody, wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   is a linking moiety or a payload;
wherein the linker is conjugated to the Gln residue comprised in the antibody via a primary amine comprised in the side chain of the lysine residue, the lysine derivative or the lysine mimetic; and
   wherein the antibody is contacted with 2.5-8 molar equivalents of the linker; and/or wherein the antibody is added to the conjugation reaction at a concentration ranging from 5-17 mg/mL; and, optionally, wherein the transglutaminase is added to the conjugation reaction at a concentration ranging from 2-10 U/mg antibody.

In another embodiment, the invention relates to a method for producing an antibody-linker conjugate by means of a transglutaminase, the method comprising a step of conjugating a linker comprising the structure (shown in N→C direction)

(Sp₁)-K-(Sp₂)-B-(Sp₃) or(Sp₁)-B-(Sp₂)-K-(Sp₃)
to a Gln residue comprised in an antibody, wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   B is a linking moiety or a payload;
wherein the linker is conjugated to the Gln residue comprised in the antibody via a primary amine comprised in the side chain of the lysine residue, the lysine derivative or the lysine mimetic; and
   wherein the antibody is contacted with 5-40 molar equivalents of the linker; and/or wherein the antibody is added to the conjugation reaction at a concentration ranging from 5-17 mg/mL; and, optionally, wherein the transglutaminase is added to the conjugation reaction at a concentration ranging from 2-10 U/mg antibody.

In another embodiment, the invention relates to a method for producing an antibody-linker conjugate by means of a transglutaminase, the method comprising a step of conjugating a linker comprising the structure (shown in N→C direction)

(Sp₁)-K-(Sp₂)-B-(Sp₃) or(Sp₁)-B-(Sp₂)-K-(Sp₃)
to a Gln residue comprised in an antibody, wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   B is a linking moiety or a payload;
wherein the linker is conjugated to the Gln residue comprised in the antibody via a primary amine comprised in the side chain of the lysine residue, the lysine derivative or the lysine mimetic; and
   wherein the antibody is contacted with 5-20 molar equivalents of the linker; and/or wherein the antibody is added to the conjugation reaction at a concentration ranging from 5-17 mg/mL; and, optionally, wherein the transglutaminase is added to the conjugation reaction at a concentration ranging from 2-10 U/mg antibody.

It is preferred within the present invention, that the linker comprises the structure (Sp₁)-K-(Sp₂)-B-(Sp₃) or (Sp₁)-B-(Sp₂)-K-(Sp₃), wherein the linker is conjugated to a glutamine residue in an antibody via a primary amine comprised in the residue K of the linker. In certain embodiment, the residue K is a lysine residue. However, in certain embodiments, the residue K may also be a lysine mimetic or a lysine derivative, provided that the lysine mimetic or lysine derivative comprises a primary amine in its amino acid side chain.

Thus, in certain embodiments, the residue K may be a lysine mimetic. The term “lysine mimetic”, as used herein, refers to a compound that has a structure that is different from lysine, but that has similar characteristics as lysine and may thus be used to replace lysine in a peptide or protein without significantly altering the function and/or structure of said peptide or protein. In certain embodiments, a lysine mimetic may differ from lysine in the length or composition of the aliphatic chain that connects the primary amine and the α-carbon atom. Thus, in certain embodiments, the lysine mimetic may be ornithine, 2,3-diaminopropionic acid, 2,4-diaminobutyric acid, 2,5-diaminopentanoic acid, 2,6-diaminohexanoic acid or 2,7-diaminoheptanoic acid. In certain embodiments, the lysine mimetic may be a beta-amino acid, such as beta-homolysine.

In certain embodiments, the residue K may be a lysine derivative. The term “lysine derivative”, as used herein, refers to a lysine or lysine mimetic, wherein one or more functional groups comprised in the lysine or lysine mimetic is (are) modified or substituted. Within the present invention, it is preferred that the amino group in the side chain of the lysine derivative is unmodified, such that is available for conjugation to a glutamine residue in a protein. In embodiments, where the residue K is located in the C-terminal position of the linker, K may be a lysine derivative wherein the α-carboxyl group is modified or substituted. In certain embodiments the α-carboxyl group of the lysine mimetic may be amidated.

Within the present invention, the lysine-based linker may have the structure (Sp₁)-K-(Sp₂)-B-(Sp₃) or (Sp₁)-B-(Sp₂)-K-(Sp₃). That is, the linker may comprise one or more chemical spacers (Sp). The term “chemical spacer” as used herein describes a chemical moiety that is covalently attached to a chemical residue of the linker and/or interposed between two chemical residues of the linker.

In a particular embodiment, the invention relates to the method according to the invention, wherein the chemical spacers (Sp₁), (Sp₂) and (Sp₃) each independently comprise between 0 and 12 amino acid residues.

That is, in certain embodiments, the chemical spacers (Sp₁), (Sp₂) and/or (Sp₃) may be present or absent. In embodiments where (Sp₁), (Sp₂) and/or (Sp₃) are present, (Sp₁), (Sp₂) and/or (Sp₃) may comprise one or more amino acid residues. In such embodiments, each of (Sp₁), (Sp₂) and/or (Sp₃) may comprise between 0 and 12 amino acid residues. It has to be noted that the chemical spacers (Sp₁), (Sp₂) and/or (Sp₃) may also comprise non-amino acid residues, which is disclosed in more detail below.

An “amino acid residue” comprised in the chemical spacer (Sp₁), (Sp₂) and/or (Sp₃) may be an amino acid, an amino acid mimetic or an amino acid derivative. It is to be understood, that the term amino acid encompasses not only α-amino acids, but also other amino acids such as β-, γ- or δ-amino acids. An α-amino acid residue may be present in the chemical spacer (Sp₁), (Sp₂) and/or (Sp₃) in its L- or D-form. In embodiments, where (Sp₁), (Sp₂) and/or (Sp₃) comprises a chiral β-, γ- or δ-amino acid, the chiral β-, γ- or δ-amino acid may be present in its S- or R-form. Thus, in its broadest sense, the term “amino acid residue”, as used herein, may refer to any organic compound that contains an amino group (—NH₂) and a carboxyl group (—COOH). Thus, whenever an “amino acid” or an “amino acid residue” is referred to throughout this disclosure, it is to be understood that the term amino acid residue may also encompass amino acid mimetics or derivatives.

Further, it is to be understood that the term amino acid residue is not limited to the known set of proteinogenic amino acids, namely alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine, but also encompasses non-canonical and non-natural amino acids. A “non-canonical amino acid”, as used herein, may be any amino acid that is not part of the set of proteinogenic amino acids, but that can be obtained from a natural source. However, it has to be noted that some non-canonical amino acids may also be found in naturally occurring peptides and/or proteins.

A “non-natural amino acid” or “synthetic amino acid”, as used herein, may be any molecule that falls under the general definition of an amino acid, i.e., that comprises an amino group and a carboxyl group, but that is not found in nature. Thus, non-natural amino acids are preferably obtained by chemical synthesis. It is to be understood that the differentiation between a non-canonical amino acid and a non-natural amino acid may be uncertain in some instances. For example, an amino acid that is defined as a non-natural amino acid may be, at a later time point, identified in nature and thus reclassified as a non-canonical amino acid.

Examples of non-canonical or non-natural amino acids may be, without limitation, D-amino acids (such as D-alanine, D-arginine, D-methionine), homo-amino acids (such as homoserine, homoarginine, homocysteine, α-aminoadipic acid), N-methylated amino acids (such as sarcosine, N-Me-leucine), α-methyl amino acids (such as α-methyl-histidine, α-aminoisobutyric acid), β-amino acids (such as β-alanine, D-3-aminoisobutyric acid, L-β-homoalanine), γ-amino acids (such as γ-aminobutyric acid), alanine mimetics or derivatives (such as β-cyclopropylalanine, phenylglycine, dehydro-alanine, β-cyanoalanine, β-(3-pyridyl)-alanine, β-(1,2,4-triazol-1-yl)-alanine, β-(1-piperazinyl)-alanine), phenylalanine mimetics or derivatives (such as 4-iodophenylalanine, pentafluoro-phenylalanine, naphthyl-alanine, 4-Aminophenylalanine), arginine mimetics or derivatives (such as β-ureidoalanine, ω-methylarginine), lysine mimetics or derivatives (such as (3-(3-methyl-3H-diazirine-3-yl)propamino)carbonyl-1-lysine, Nε,Nε,Nε-trimethyllysine), histidine mimetics or derivatives (such as 2,5-di-iodohistidine, 1-methylhistidine), tyrosine mimetics or derivatives (such as 3-aminotyrosine, thyronine, 3,5-dinitrotyrosine, 3-hydroxy-methyl-tyrosine, O-phospho-L-tyrosine), tryptophan mimetics or derivatives (such as 5-hydroxy-tryptophan, 1-methyltryptophan), serine mimetics or derivatives (such as β-(2-thienyl)-serine, β-(3,4-dihydroxyphenyl)-serine, 0-phosphoserine), threonine mimetics or derivatives (such as allo-threonine, O-phosphothreonine), proline mimetics or derivatives (such as Hydroxyproline, 3,4-dehydro-proline, pyroglutamic acid, thiaproline, cis-octahydroindole-2-carboxylic acid), leucine and isoleucine mimetics or derivatives (such as allo-isoleucine, norleucine, 4,5-dehydroleucine, (4S)-4-hydroxy-L-isoleucine), valine mimetics or derivatives (such as norvaline, γ-hydroxyvaline), citrulline mimetics or derivatives (such as thiocitrulline, homocitrulline), cysteine mimetics or derivatives (such as penicillamine, selenocysteine, buthionine-sulfoximine), methionine mimetics or derivatives (such as S-methylmethionine, L-methionine sulfone, L-methionine sulfoxide, L-methionine sulfoximine, selenomethionine), aspartic acid mimetics or derivatives (such as DL-threo-p-hydroxyaspartic acid, L-aspartic acid β-methyl ester), glutamic acid mimetics or derivatives (such as γ-methyleneglutamic acid, γ-carboxyglutamic acid, γ-hydroxyglutamic acid, L-glutamic acid 5-methyl ester, L-2-aminoheptanedioic acid), asparagine mimetics or derivatives (such as L-threo-3-hydroxyasparagine, N,N-dimethyl-L-asparagine, L-2-amino-2-carboxyethanesulfonamide, 5-diazo-4-oxo-L-norvaline), glutamine mimetics or derivatives (such as 4-F-(2S,4R)-fluoroglutamine, γ-glutamylmethylamide, theanine, L-glutamic acid γ-monohydroxamate), amino acids comprising a cyclic moiety (such as 4-aminopiperidine-4-carboxylic acid, azetidine-2-carboxylic acid, pipecolic acid, 1-aminocyclopentanecarboxylic acid, spinacine), or amino acids comprising a bio-orthogonal moiety (such as propargylglycine, α-allylglycine, L-azido-homoalanine, p-benzoyl-1-phenylalanine, p-2-fluoroacetyl-1-phenylalanine, (S)-2-amino-3-(4-(6-methyl-1,2,4,5-tetrazin-3-yl)phenyl) propanoic acid).

Besides the alpha-amino acids described above, the chemical spacers (Sp₁), (Sp₂) and/or (Sp₃) may comprise one or more β-, γ-, δ- or ε-amino acids. Thus, in certain embodiments, the linker may be a peptidomimetic. The peptidomimetic may not exclusively contain classical peptide bonds that are formed between two α-amino acids but may additionally or instead comprise one or more amide bonds that are formed between an alpha amino acid and a β-, γ-, δ- or ε-amino acid, or between two β-, γ-, δ- or ε-amino acids, respectively. Accordingly, in any instance of the present invention where the linker is described as a peptide, it is to be understood that the linker may also be a peptidomimetic and thus not exclusively consist of α-amino acids, but may instead comprise one or more β-, γ-, δ- or ε-amino acids or molecules that are not classified as an amino acid. Examples of β-, γ-, δ- or ε-amino acids that may be comprised in the linker of the present invention include, but are not limited to, β-alanine, γ-aminobutyric acid, 4-amino-3-hydroxy-5-phenylpentanoic acid, 4-amino-3-hydroxy-6-methylheptanoic acid, 6-aminohexanoic acid and statine.

Furthermore, the chemical spacers (Sp₁), (Sp₂) and/or (Sp₃) may comprise amino acid derivatives and/or amino acid mimetics. In embodiments where (Sp₁), (Sp₂) and/or (Sp₃) comprise one or more amino acid derivatives, it is preferred that the amino acid derivatives have free amino and carboxyl groups, such that they can undergo the formation of peptide or isopeptide bonds. In embodiments where (Sp₁), (Sp₂) and/or (Sp₃) comprise one or more amino acid mimetics, the amino acid mimetics may have free amino and carboxyl groups, such that they can undergo the formation of peptide or isopeptide bonds. However, in certain embodiments, amino acid mimetics or derivatives may have a substituted amino group that does not prevent the formation of a peptide bond. Examples of such amino acid mimetics or derivatives may be N-methylated amino acids such as sarcosine or N-Me-leucine.

In embodiments where an amino acid residue comprised in (Sp₁) or (Sp₃) is a terminal amino acid residue, the terminal amino acid residue may comprise a modified, protected or substituted N-terminal amino group or C-terminal carboxyl group.

Further, the amino acid mimetic or derivative may be an amino acid comprising a derivatized amino group, such as mimetics or derivatives of proline or other cyclic amino acids such as azetidine-2-carboxylic acid, pipecolic acid or spinacine. Further, an amino acid mimetic may also comprise other functional groups that replace the amino and/or carboxyl groups of a standard amino acid, which allows the amino acid mimetic to undergo the formation of alternative bonds with adjacent amino acids, amino acid derivatives and/or amino acid mimetics and to form a peptidomimetic.

The term “amino acid mimetic”, as used herein, refers to a compound that has a structure that is different from a particular amino acid, but that functions in a manner similar to said particular amino acid and may thus be used to replace said particular amino acid. An amino acid mimetic is said to function in a similar manner as a particular amino acid, if it fulfils, at least to some extent, similar structural and/or functional features as the amino acid it mimics.

The term “amino acid derivative” refers to an amino acid as defined herein, wherein one or more functional group(s) comprised in the amino acid is (are) modified or substituted. An amino acid derivative may preferably be a derivative of a proteinogenic or non-canonical amino acid. Any functional group of an amino acid derivative may be substituted or modified.

In embodiments where the linker comprises one or more terminal amino acid residue(s), the terminal amino acid residue may be protected. For example, in embodiments where (Sp₁) comprises an N-terminal amino acid residue, the N-terminal amino group may be protected. For example, in certain embodiments, the N-terminal amino acid residue comprised in the spacer (Sp₁) may be acetylated. In other embodiments, the lysine residue K may be the N-terminal amino acid of the linker. In such embodiments, the N-terminal amino group of the lysine, the lysine mimetic or the lysine derivative may be protected, for example by acetylation. In certain embodiments, the linking moiety B or the payload B may be an amino acid or based on an amino acid. In such embodiments, the N-terminal amino group of the amino acid-based payload or linking moiety B may be protected, for example by acetylation.

Likewise, in embodiments where (Sp₃) comprises a C-terminal amino acid residue, the C-terminal carboxyl group may be protected. For example, in certain embodiments, the C-terminal amino acid residue in the spacer (Sp₃) may be amidated. In other embodiments, the K residue may be the C-terminal amino acid of the linker. In such embodiments, the C-terminal carboxyl group of the lysine, the lysine mimetic or the lysine derivative may be protected, for example by amidation. In certain embodiments, the linking moiety B or the payload B may be an amino acid or based on an amino acid. In such embodiments, the C-terminal carboxyl group of the amino acid-based payload or linking moiety B may be protected, for example by amidation.

In certain embodiments, each of the chemical spacers (Sp₁), (Sp₂) and/or (Sp₃) may comprise 0 to 12 amino acid residues, including amino acid derivatives and amino acid mimetics. That is, in certain embodiments, (Sp₁) may comprise 0, 1, 2, 3, 4, 5, 6, 7, 8, 9, 10, 11 or 12 amino acid residues, (Sp₂) may comprise 0, 1, 2, 3, 4, 5, 6, 7, 8, 9, 10, 11 or 12 amino acid residues and (Sp₃) may comprise 0, 1, 2, 3, 4, 5, 6, 7, 8, 9, 10, 11 or 12 amino acid residues.

In a particular embodiment, the invention relates to the method according to the invention, wherein the linker comprises not more than 25, 20, 15, 14, 13, 12, 11, 10, 9, 8, 7, 6, 5, 4 amino acid residues.

That is, in certain embodiments, the linker may comprise 25, 24, 23, 22, 21, 20, 19, 18, 17, 16, 15, 14, 13, 12, 11, 10, 9, 8, 7, 6, 5, 4, 3 or 2 amino acid residues, including amino acid mimetics and amino acid derivative. It is to be understood that the amino acid residues comprised in the linker, including amino acid mimetics and amino acid derivatives, are preferably amino acid residues comprised in the K residue, in the chemical spacers (Sp₁), (Sp₂) and/or (Sp₃) and, in certain embodiments, also in B, when B is an amino acid-based linking moiety or payload.

In certain embodiments, the linker may comprise between 2 and 25 amino acid residues, including amino acid mimetics and amino acid derivatives. In other embodiments, the linker may comprise between 2 and 20 amino acid residues, including amino acid mimetics and amino acid derivatives. In other embodiments, the linker may comprise between 2 and 15 amino acid residues, including amino acid mimetics and amino acid derivatives. In other embodiments, the linker may comprise between 2 and 10 amino acid residues, including amino acid mimetics and amino acid derivatives. In other embodiments, the linker may comprise between 3 and 10 amino acid residues, including amino acid mimetics and amino acid derivatives. In other embodiments, the linker may comprise between 3 and 8 amino acid residues, including amino acid mimetics and amino acid derivatives. In other embodiments, the linker may comprise between 3 and 6 amino acid residues, including amino acid mimetics and amino acid derivatives.

In a particular embodiment, the invention relates to the method according to the invention, wherein the net charge of the linker is neutral or positive.

In certain embodiments, the linker is a peptide linker (or a peptidomimetic as disclosed herein). That is, the chemical spacers (Sp₁), (Sp₂) and/or (Sp₃), if present, consist exclusively of amino acids, amino acid mimetics or amino acid derivatives. The net charge of a peptide is usually calculated at neutral pH (7.0). In the simplest approach, the net charge is determined by adding the number of positively charged amino acid residues (Arg and Lys and optionally His) and the number of negatively charged ones (Asp and Glu) and calculate the difference of the two groups. In cases where the linker comprises non-canonical amino acids or amino acid derivatives in which a charged functional group is modified or substituted, the skilled person is aware of methods to determine the charge of the non-canonical amino acid or amino acid derivative at neutral pH.

In certain embodiments, the payload or linking moiety B or any non-amino acid moiety comprised in (Sp₁), (Sp₂) and/or (Sp₃) may also contribute to the net charge of the linker.

However, the skilled person is aware of methods to calculate the net charge of the entire linker, including any non-amino acid moieties, preferably at neutral pH (7.0).

In certain embodiments, the net charge of a linker is calculated solely based on the amino acid residues comprised in the linker, including amino acid mimetics and amino acid derivatives.

Thus, in a particular embodiment, the invention relates to the method according to the invention, wherein the net charge of the amino acid residues comprised in the linker is neutral or positive.

In a particular embodiment, the invention relates to the method according to the invention, wherein the linker comprises no negatively-charged amino acid residues.

That is, the linker may be free of negatively charged amino acid residues, including amino acid mimetics and amino acid derivatives. A negatively charged amino acid residue is an amino acid, amino acid mimetic or amino acid derivative which carries a negative charge at neutral pH (7.0). Negatively charged canonical amino acids are glutamic acid and aspartic acid. However, negatively charged non-canonical amino acids, amino acid mimetics and amino acid derivatives are known in the art.

In a particular embodiment, the invention relates to the method according to the invention, wherein the linker comprises at least one positively-charged amino acid residue besides residue K. That is, (Sp₁), (Sp₂) and/or (Sp₃) may comprise at least one positively-charged amino acid. In certain embodiments, (Sp₁), (Sp₂) and/or (Sp₃) comprise at least one histidine or arginine residue. However, it is preferred herein that the linker does not comprise the sequence motif RK, wherein R is arginine, an arginine mimetic or an arginine derivative and wherein K is lysine, a lysine mimetic or a lysine derivative. That is, in a particularly preferred embodiment, the invention relates to a method for producing an antibody-linker conjugate by means of a transglutaminase, the method comprising a step of conjugating a linker comprising the structure (shown in N→C direction)

(Sp₁)-K-(Sp₂)-B-(Sp₃) or(Sp₁)-B-(Sp₂)-K-(Sp₃)
to a Gln residue comprised in an antibody, wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   B is a linking moiety or a payload;
wherein the linker is conjugated to the Gln residue comprised in the antibody via a primary amine comprised in the side chain of the lysine residue, the lysine derivative or the lysine mimetic;
wherein the antibody is contacted with less than 80 molar equivalents of the linker; and
wherein the residue K is not directly coupled to an N-terminal arginine, arginine mimetic or arginine derivative.

In certain embodiments, (Sp₁), (Sp₂) and/or (Sp₃) comprise at least one histidine residue. In certain embodiments, the histidine residue is directly linked to the lysine residue, the lysine mimetic or the lysine derivative. That is, in certain embodiments, the linker according to the invention comprises the motif HK.

In a particular embodiment, the invention relates to a method for producing an antibody-linker conjugate by means of a transglutaminase, the method comprising a step of conjugating a linker comprising or consisting of the structure (shown in N→C direction)

(Sp₁)-K-(Sp₂)-B-(Sp₃) or (Sp₁)-B-(Sp₂)-K-(Sp₃)
to a Gln residue comprised in an antibody, wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   B is a linking moiety or a payload;
wherein the linker is conjugated to the Gln residue comprised in the antibody via a primary amine comprised in the side chain of the lysine residue, the lysine derivative or the lysine mimetic;
wherein the antibody is contacted with less than 80 molar equivalents of the linker; and
wherein the linker comprises at least one histidine residue, in particular wherein the at least one histidine residue is directly linked to the residue K, in particular wherein the linker comprises the sequence motif HK.

In a particular embodiment, the invention relates to an antibody conjugate comprising a linker comprising or consisting of the structure (shown in N→C direction)

(Sp₁)-K-(Sp₂)-B-(Sp₃) or(Sp₁)-B-(Sp₂)-K-(Sp₃)
wherein
   (Sp₁) is a chemical spacer or is absent;
   (Sp₂) is a chemical spacer or is absent;
   (Sp₃) is a chemical spacer or is absent;
   K is lysine or a lysine derivative or a lysine mimetic;
   B is a linking moiety or a payload;
wherein the linker is conjugated to a Gln residue comprised in the antibody via a primary amine comprised in the side chain of the lysine residue, the lysine derivative or the lysine mimetic; and
wherein the linker comprises at least one histidine residue, in particular wherein the at least one histidine residue is directly linked to the residue K, in particular wherein the linker comprises the sequence motif HK.

Besides or instead of amino acid residues, including amino acid mimetics and derivatives, the chemical spacers (Sp₁), (Sp₂) and/or (Sp₃) may comprise or consist of non-amino acid moieties.

That is, in certain embodiments, the chemical spacers (Sp₁), (Sp₂) and/or (Sp₃) may not exclusively consist of amino acids, amino acid mimetics or amino acid derivatives. That is, the chemical spacers (Sp₁), (Sp₂) and/or (Sp₃) may comprise non-amino acid components or may exclusively consist of non-amino acid components. In certain embodiments, the chemical spacers (Sp₁), (Sp₂) and/or (Sp₃) may comprise amino acid and non-amino acid components.

For example, but without limitation, each of the chemical spacers (Sp₁), (Sp₂) and/or (Sp₃) may comprise a carbon comprising framework of 1 to 200 atoms, optionally a carbon comprising framework of at least 10 atoms, e.g. 10 to 100 atoms or 20 to 100 atoms, substituted at one or more atoms, optionally wherein the carbon comprising framework is a linear hydrocarbon or comprises a cyclic group, a symmetrically or asymmetrically branched hydrocarbon, monosaccharide, disaccharide, linear or branched oligosaccharide (asymmetrically branched or symmetrically branched), other natural linear or branched oligomers (asymmetrically branched or symmetrically branched), or more generally any dimer, trimer, or higher oligomer (linear, asymmetrically branched or symmetrically branched) resulting from any chain-growth or step-growth polymerization process.

(Sp₁), (Sp₂) and/or (Sp₃) may be any straight, branched and/or cyclic C₂₋₃₀ alkyl, C₂₋₃₀ alkenyl, C₂₋₃₀ alkynyl, C₂₋₃₀ heteroalkyl, C₂₋₃₀ heteroalkenyl, C₂₋₃₀ heteroalkynyl, optionally wherein one or more homocyclic aromatic compound radical or heterocyclic compound radical may be inserted; notably, any straight or branched C₂₋₅ alkyl, C₅₋₁₀ alkyl, C₁₁₋₂₀ alkyl, —O—C₁₋₅ alkyl, —O—C₅₋₁₀ alkyl, —O—C₁₁₋₂₀ alkyl, or (CH₂—CH₂—O—)₁₋₂₄ or (CH₂)ₓ₁—(CH₂—O—CH₂)₁₋₂₄—(CH₂)ₓ₂— group, wherein x1 and x2 are independently an integer selected among the range of 0 to 20, an amino acid, an oligopeptide, glycan, sulfate, phosphate, or carboxylate. In some embodiments, (Sp₁), (Sp₂) and/or (Sp₃) may comprise a C₂₋₆ alkyl group.

In certain embodiments, the chemical spacers (Sp₁), (Sp₂) and/or (Sp₃) may comprise one or more polyethylene glycol (PEG) moieties or comparable condensation polymers, such as poly(carboxybetaine methacrylate) (pCBMA), polyoxazoline, polyglycerol, polyvinylpyrrolidone or poly(hydroxyethylmethacrylate) (pHEMA). Polyethylene glycol (PEG) is a polyether compound with many applications from industrial manufacturing to medicine. PEG is also known as polyethylene oxide (PEO) or polyoxyethylene (POE), depending on its molecular weight. The structure of PEG is commonly expressed as H—(O—CH₂—CH₂)ₙ—OH. The skilled person is aware of methods to functionalize condensation polymers such that they can be coupled to an amino acid residue or a payload.

Thus, in a particular embodiment, the invention relates to the method according to the invention, wherein the linker comprises one or more PEG moieties. In certain embodiments, a PEG moiety may be comprised in the chemical spacers (Sp₁), (Sp₂) and/or (Sp₃). In certain embodiments, each PEG moiety comprised in a linker may comprise between 2 and 20 ethylene glycol monomers, between 2 and 15 ethylene glycol monomers, between 2 and 10 ethylene glycol monomers or between 2 and 5 ethylene glycol monomers. In certain embodiments, a PEG moiety is comprised in (Sp₂) to directly connect a linking moiety or a payload to the K residue. In certain embodiments, a PEG moiety is comprised in (Sp₂) to connect a linking moiety or a payload to an amino acid residue comprised in (Sp₂). In certain embodiments, a PEG moiety is comprised in (Sp₂) to connect the K residue to a self-immolative moiety, which is in turn connected to a payload. In certain embodiments, a PEG moiety is comprised in (Sp₂) to connect an amino acid residue comprised in (Sp₂) with a self-immolative moiety, which is in turn connected to a payload.

In certain embodiments, the chemical spacers (Sp₁), (Sp₂) and/or (Sp₃) may comprise a dextran. The term “dextran” as used herein refers to a complex, branched glucan composed of chains of varying lengths, which may have weights of ranging from 3 to 2000 kDa. The straight chain typically consists of alpha-1,6 glycosidic linkages between glucose molecules, while branches begin from alpha-1,3 linkages. Dextran may be synthesized from sucrose, e.g. by lactic acid bacteria. In the context of the present invention dextran to be used as carrier may preferably have a molecular weight of about 15 to 1500 kDa.

In certain embodiments, the chemical spacers (Sp₁), (Sp₂) and/or (Sp₃) may comprise an oligonucleotide. The term “oligonucleotide” as used herein refers to an oligomer or polymer of either ribonucleic acid (RNA) or deoxyribonucleic acid (DNA), as well as non-naturally occurring oligonucleotides. Due to higher stability, an oligonucleotide is preferably a polymer of DNA.

In certain embodiments, the chemical spacers (Sp₁), (Sp₂) and/or (Sp₃), if present, consist exclusively of amino acid residues, including amino acid mimetics and derivatives, and PEG moieties. In certain embodiments, the chemical spacers (Sp₁), (Sp₂) and/or (Sp₃), if present, consist exclusively of amino acid residues, including amino acid mimetics and derivatives. In certain embodiments, all amino acid residues comprised in the chemical spacers (Sp₁), (Sp₂) and/or (Sp₃) are α-L-amino acids. That is, in certain embodiments, the linker, excluding the payload or linking moiety B, consists exclusively of amino acid residues. In certain embodiments, the linker, excluding the payload or linking moiety B, consists exclusively of α-L-amino acid residues. Such peptide-based linkers may comprise a protection group at the N- and/or C-terminus. That is, the N-terminal amino group may be acetylated and/or the C-terminal carboxyl group may be amidated.

It has to be noted that the chemical spacers (Sp₁), (Sp₂) and/or (Sp₃) may have identical structures. However, it is preferred that each of the chemical spacers (Sp₁), (Sp₂) and/or (Sp₃) has a different structure and/or that not all of the chemical spacers (Sp₁), (Sp₂) and/or (Sp₃) are present at the same time. That is, in certain embodiments, only one or two of the chemical spacers (Sp₁), (Sp₂) and/or (Sp₃) may be present in a linker.

In certain embodiments, the K residue may be directly connected to one or more small hydrophobic amino acid residue. For example, in certain embodiments, the K residue may be directly connected to one or more alanine residues.

Within the present invention, it is preferred that the linker is conjugated to the antibody via a primary amine comprised in the side chain of the residue K. Thus, it is preferred that the chemical spacers (Sp₁), (Sp₂) and/or (Sp₃) do not comprise additional lysine residues, lysine mimetics or lysine derivatives that could serve as additional amine donors in the transglutaminase-based conjugation reaction. In other embodiments, any free N-terminal amino group comprised in a linker may be substituted, e.g., acetylated, such that it cannot serve as substrate for a transglutaminase.

The linker according to the invention further comprises at least one linking moiety or payload B. The linker according to the invention may be used to directly conjugate a payload to an antibody in a one-step conjugation process. In other embodiments, a linker comprising one or more linking moiety may be conjugated to an antibody in a first step and one or more payloads may then be linked to the antibody-linker conjugate in a second step. The following Table 1 clarifies the two terms as used herein:

TABLE 1
One- and two step conjugation

| Linker peptide (exemplary) | Process type | Steps |
|---|---|---|
| (Sp₁)-K-(Sp₂)-payload-(Sp₃) or | One-step | step 1: conjugation of |
| (Sp₁)-payload-(Sp₂)-K-(Sp₃) | conjugation | linker comprising the |
| | | payload to Gln residue |
| | | in antibody |
| (Sp₁)-K-(Sp₂)-linking | Two-step | step 1: conjugation of |
| moiety-(Sp₃) or (Sp₁)-linking | conjugation | linker comprising the |
| moiety-(Sp₂)-K-(Sp₃) | | linking moiety to Gln |
| | | residue in antibody |
| | | step 2: conjugation of a |
| | | payload to linking moiety |

In certain embodiments, the linker may comprise one or more linking moieties B. Thus, in a particular embodiment, the invention relates to the method according to the invention, wherein B is a linking moiety.

A “linking moiety” as used herein generally refers to an at least bi-functional molecule. Within the present invention, a linking moiety comprises a first functional group that allows coupling of the linking moiety to the linker of the invention and a second functional group that can be used for coupling an additional molecule to the linker before or after the linker has been conjugated to an antibody. In certain embodiments, the linking moiety of the invention is an amino acid, an amino acid mimetic or an amino acid derivative. In such embodiments, the linking moiety is preferably connected to the linker via its amino group, while the functional group comprised in the amino acid side chain can be used for coupling an additional molecule to the linker. Alternatively, the linking moiety may be connected to the linker via its carboxyl group, while the functional group comprised in the amino acid side chain can be used for coupling an additional molecule to the linker.

In a particular embodiment, the invention relates to the method according to the invention, wherein the linking moiety B comprises
a bioorthogonal marker group, or
a non-bio-orthogonal entity for crosslinking.

The term “bioorthogonal marker group” has been established by Sletten and Bertozzi (A Bioorthogonal Quadricyclane Ligation. J Am Chem Soc 2011, 133 (44), 17570-17573) to designate reactive groups that can lead to chemical reactions to occur inside of living systems without interfering with native biochemical processes. A “non-bio-orthogonal entity for crosslinking” may be any molecule that comprises or consists of a first functional group, wherein the first functional group can be chemically or enzymatically crosslinked to a payload comprising a compatible second functional group. Even in cases where the crosslinking reaction is a non-bio-orthogonal reaction, it is preferred that the reaction does not introduce additional modifications to the antibody other than the crosslinking of the payload to the linker. In view of the above, the linking moiety B may either consist of the “bioorthogonal marker group” or the “non-bio-orthogonal entity” or may comprise the “bioorthogonal marker group” or the “non-bio-orthogonal entity”. For example, in case of the linking moiety Lys(N₃), both the entire Lys(N₃) and the azide group alone may be seen as a bioorthogonal marker group within the present invention. Lys(N₃) refers to 6-azido-L-lysine, which may also be abbreviated K(N₃).

In a particular embodiment, the invention relates to the method according to the invention, wherein the bioorthogonal marker group or the non-bio-orthogonal entity for crosslinking consists of or comprises at least one molecule or moiety selected from the group consisting of:
—N—N≡N, or —N₃;
Lys(N₃);
a tetrazine;
an alkyne;
a strained cyclooctyne;
BCN;
a strained alkene;
a photoreactive group;
an aldehyde;
an acyltrifluoroborate;
a protein degradation agent (‘PROTAC’);
a cyclopentadiene/spirolocyclopentadiene;
a thio-selective electrophile;
—SH; and
cysteine.

The bioorthogonal marker group or the non-bio-orthogonal entity for crosslinking comprised in the linker may, for example, engage in any of the binding reactions shown in Table 2:

TABLE 2

| binding partner 1 | binding partner 2 | reaction type |
|---|---|---|
| —N—N≡N | cyclooctyne derivatives (e.g. | SPAAC |
| | DIFO, BCN, DIBAC, DIBO, | |
| | ADIBO/DBCO) | |
| —N—N≡N | Alkyne | CuAAC |
| —N—N≡N | Triarylphosphines | Staudinger ligation |
| tetrazine | Cyclopropene | tetrazine ligation |
| | Norborene | |
| | Trans-cyclooctene | |
| | Cyclooctyne | |
| | (BCN) | |
| —SH, e.g., of a Cys residue | Maleimide | Thiol-Maleimide |
| | | conjugation |
| Amine | N-hydroxysuccinimid | |
| —O— | Acyltrifluoroborates | KAT-ligation (potassium |
| carbamoylhydroxylamines 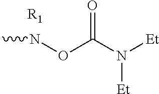 | 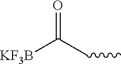 | acyl-trifluoroborate) |
| Rₓ—S—S—R_{y} | R_{z}—SH + reducing agent (e.g. TCEP, | Direct disulfide |
| | DTT) | bioconjugation |
| —CHO (aldehyde) | HIPS-probe | Hydrazino-iso- |
| | | Pictet- |
| | 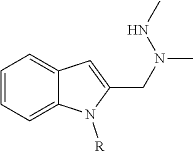 | Spengler (HIPS) |
| —CHO (aldehyde) | N-pyrrolyl alanine derivative | pyrrolyl alanine Pictet- |
| | | Spengler (PAPS) |
| —CHO (aldehyde) | R₁—N—N—R₂ | Hydrazone-ligation |
| | HO—N—R₁ | Oxime-ligation |
| | H2N—CHR₁—CH2—SH | Thiazolidine-Ligation |
| maleimide | —SH, e.g., of a Cys residue | Thiol-Maleimide |
| | | conjugation |
| maleimide | 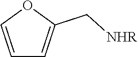 | Thiol-cylcopentadiene conjugation (Diels-Alder Reaction) |
| | 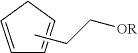 | |
| | 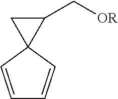 | |
| | 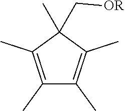 | |
| Biotin | Streptavidin | Biotin-streptavidin |
| | | interaction |

The linking moiety B may either be or comprise what is called “binding partner 1” or “binding partner 2” in Table 2.

In certain embodiments, the linking moiety B may be a cysteine, a cysteine mimetic or a cysteine derivative with a free sulfhydryl group.

The free sulfhydryl group of such Cys residue (or mimetic or derivative) may be conjugated to a payload construct comprising a thio-selective electrophile, such as maleimide. Toxin constructs comprising a maleimide moiety have frequently been used, and also approved by medical authorities, like Adcetris. Thus, toxin constructs comprising an MMAE toxin may be coupled to a free sulfhydryl group of a Cys residue in the linker of the invention.

It has to be noted that also other thio-selective electrophiles such as 3-arylpropionitrile (APN) or phosphonamidate may be used instead of maleimide in the method of the invention.

Providing a Cys-residue in the linker according to the present invention does therefore have the advantage to allow using off-the-shelf-toxin-maleimide constructs to create antibody-payload conjugates, or, more generally, to be able to fully exploit the advantages of Cys-maleimide binding chemistry. At the same time, off-the-shelf antibodies can be used, which do not have to be deglycosylated. In specific embodiments, the Cys residue may be C-terminal or intrachain in the amino acid-based linker.

In another embodiment, the linking moiety B may comprise an azide group. The skilled person is aware of molecules comprising an azide group which may be incorporated into a linker according to the invention, such as 6-azido-lysine (Lys(N₃)) or 4-azido-homoalanine (Xaa(N₃)). Linking moieties comprising an azide group may be used as substrates in various bio-orthogonal reactions, such as strain-promoted azide-alkyne cycloaddition (SPAAC), copper-catalyzed azide-alkyne cycloaddition (CuAAC) or Staudinger ligation. For example, in certain embodiments, payloads comprising a cyclooctyne derivative, such as DBCO, DIBO, BCN or BARAC may be coupled to a linker comprising an azide group by SPAAC.

In yet another embodiment, the linking moiety B may comprise a tetrazine group. The skilled person is aware of tetrazine-comprising molecules which may be incorporated into a linker according to the invention, preferably amino acid derivatives comprising a tetrazine group.

Linking moieties comprising a tetrazine may be used as substrates in a bio-orthogonal tetrazine ligation. For example, in certain embodiments, payloads comprising a cyclopropene, a norborene, a norborene derivative or a cyclooctyne group, such as bicyclo[6.1.0]nonyne (BCN), may be coupled to a linker comprising a tetrazine group.

In certain embodiments, the linking moiety B may comprise a cyclic diene, such as a cyclopentadiene derivative. Potential cyclopentadienes derivatives that can be linked to a maleimide-comprising payload molecule have been described by Amant et al., Tuning the Diels-Alder Reaction for Bioconjugation to Maleimide Drug-Linkers; Bioconjugate Chem. 2018, 29, 7, 2406-2414 and Amant et al., A Reactive Antibody Platform for One-Step Production of Antibody-Drug Conjugates through a Diels-Alder Reaction with Maleimide; Bioconjugate Chem. 2019, 30, 9, 2340-2348.

In certain embodiments, the linking moiety B may comprise a photoreactive group. The term “photoreactive group”, as used herein, refers to a chemical group that responds to an applied external energy source in order to undergo active species generation, resulting in covalent bonding to an adjacent chemical structure (e.g., an abstractable hydrogen). Examples of photoreactive groups are, without limitation, aryl azides, such as phenyl azide, o-hydroxyphenyl azide, m-hydroxyphenylazide, tetrafluorophenyl azide, o-nitrophenyl azide, m-nitrophenyl azide, or azido-methylcoumarin, diazirine, psoralen or benzophenon.

In a particular embodiment, the invention relates to the method according to the invention, the method comprising a further step of conjugating one or more payloads to the linking moiety B.

Instead of directly conjugating a linker comprising one or more payloads to an antibody in a one-step process, the invention, in certain embodiments, refers to a two-step process, wherein a linker comprising at least one linking moiety B is conjugated to an antibody in a first step and one or more payloads may be subsequently coupled to the linking moiety B in a second step.

The term “payload”, as used herein, represents any naturally occurring or synthetically generated molecule, including small-molecular weight molecules or chemical entities that can chemically be synthesized, and larger molecules or biological entities that need to be produced by fermentation of host cells or may also be synthesized chemically and that confer a novel functionality to an antibody. It is to be understood that the payload may comprise further structures or functional groups that allow coupling of the payload to a linking moiety comprised in a linker or to other parts of the linker, such as the chemical spacers (Sp₁) and/or (Sp₃) or the K residue.

In a two-step conjugation process, a payload may be linked to a linking moiety by any suitable method known in the art. Preferably, the payload may be linked to any of the bioorthogonal marker groups or non-bio-orthogonal entities for crosslinking that have been disclosed herein. That is, the payload preferably comprises a functional group that is compatible with a bioorthogonal marker group or non-bio-orthogonal entities for crosslinking comprised in the at least one linking moiety B.

Several bioorthogonal reactions that may be used for linking a payload to a bioorthogonal marker group comprised in a linking moiety B are known in the art. For example, a number of chemical ligation strategies have been developed that fulfill the requirements of bio-orthogonality, including the 1,3-dipolar cycloaddition between azides and cyclooctynes (also termed copper-free click chemistry, Baskin et al (“Copper-free click chemistry for dynamic in vivo imaging”. Proceedings of the National Academy of Sciences. 104 (43): 16793-7)), between nitrones and cyclooctynes (Ning et al (“Protein Modification by Strain-Promoted Alkyne-Nitrone Cycloaddition”. Angewandte Chemie International Edition. 49 (17): 3065)), oxime/hydrazone formation from aldehydes and ketones (Yarema, et al (“Metabolic Delivery of Ketone Groups to Sialic Acid Residues. Application To Cell Surface Glycoform Engineering”. Journal of Biological Chemistry. 273 (47): 31168-79)), the tetrazine ligation (Blackman et al (“The Tetrazine Ligation: Fast Bioconjugation based on Inverse-electron-demand Diels-Alder Reactivity”. Journal of the American Chemical Society. 130 (41): 13518-9)), the isonitrile-based click reaction (Stöckmann et al (“Exploring isonitrile-based click chemistry for ligation with biomolecules”. Organic & Biomolecular Chemistry. 9 (21): 7303)), and most recently, the quadricyclane ligation (Sletten & Bertozzi (JACS, A Bioorthogonal Quadricyclane Ligation. J Am Chem Soc 2011, 133 (44), 17570-17573)), Copper(I)-catalyzed azide-alkyne cycloaddition (CuAAC, Kolb & Sharpless (“The growing impact of click chemistry on drug discovery”. Drug DiscovToday. 8 (24): 1128-1137)), Strain-promoted azide-alkyne cycloaddition (SPAAC, Agard et al (“A Comparative Study of Bioorthogonal Reactions with Azides”. ACS Chem. Biol. 1: 644-648)), or Strain-promoted alkyne-nitrone cycloaddition (SPANC, MacKenzie et al (“Strain-promoted cycloadditions involving nitrones and alkynes-rapid tunable reactions for bioorthogonal labeling”. Curr Opin Chem Biol. 21: 81-8)). All these documents are incorporated by reference herein to provide sufficient enabling disclosure, and avoid lengthy repetitions.

It is to be understood that the payload is preferably coupled to the bio-orthogonal marker group or the non-bio-orthogonal entity for crosslinking comprised in the linker according to the invention after said linker has been conjugated to a Gln residue of an antibody by means of a transglutaminase. However, the invention also encompasses antibody-linker conjugates wherein one or more payloads have been coupled to a linker comprising at least one linking moiety B in a first step and wherein the resulting linker-payload construct is conjugated to the antibody by a transglutaminase in a second step.

In a particular embodiment, the invention relates to the method according to the invention, wherein the one or more payload is conjugated to the linking moiety B via a click-reaction.

That is, one or more payloads may be linked to a linking moiety B in a click-reaction, in particular any of the click reaction disclosed herein.

In a particularly preferred embodiment, at least one payload may be conjugated to the linking moiety B comprised in a linker via a thiol-maleimide conjugation. That is, in certain embodiments, the payload may comprise a maleimide group and the linking moiety B may be a molecule comprising a thiol group, such as, without limitation, a cysteine residue or a cysteine mimetic such as homocysteine. However, B may also be a non-amino acid molecule comprising a free thiol group. In another embodiment, the payload may comprise a free thiol group and the linking moiety B may comprise a maleimide group.

In another particularly preferred embodiment, at least one payload may be conjugated to the linking moiety B comprised in a linker via strain-promoted azide-alkyne cycloaddition (SPAAC).

That is, in certain embodiments, the payload may comprise an alkyne group, such as, without limitation, a cycloocytne group, and the linking moiety B may be a molecule comprising an azide group, such as, without limitation, the lysine derivative Lys(N₃) disclosed herein.

However, B may also be non-amino acid molecules comprising a free azide group. In another embodiment, the payload may comprise an alkyne group, such as a cyclooctyne group and the linking moiety B may comprise an azide group.

Besides a click reaction between the linking moiety in the linker and a functional group in the payload, the payload may be covalently bound to the linking moiety by any enzymatic or non-enzymatic reaction known in the art.

It is preferred that a payload is linked to a linking moiety via a covalent bond. However, in certain embodiments, a payload may be linked to a linking moiety via a strong non-covalent bond. That is, in certain embodiments, the linking moiety B may comprise a biotin moiety, such as, without limitation, the lysine derivative biocytin. In such embodiments, a payload comprising a streptavidin moiety may be linked to the linker comprising a biotin moiety.

In a particular embodiment, the invention relates to the method according to the invention, wherein B is a payload.

In certain embodiments, the payload may already be part of the linker such that the payload can be conjugated to the antibody in a one-step process. In such embodiments, the linker is preferably coupled to the linker by chemical synthesis. The payload is preferably coupled to a chemical spacer comprised in the linker or directly to the K residue. In embodiments where the payload is coupled to an amino acid residue, including amino acid mimetics and derivatives, the payload may be coupled to the C-terminal carboxyl group or the N-terminal amino group of an amino acid residue. Alternatively, the payload may be coupled to a functional group comprised in the side chain of an amino acid residue. The skilled person is aware of methods to functionalize a payload such that it can be coupled to a carboxyl group, an amino group or an amino acid side chain.

Further, the skilled person is aware of methods to couple a payload to an amino acid-based linker by chemical synthesis. For example, an amine-comprising payload, or a thiol-comprising payload (for e.g. maytansine analogs), or a hydroxyl-containing payload (for e.g. SN-38 analogs) may be attached to the C-terminus of an amino acid-based linker by chemical synthesis. However, the skilled person is aware of further reactions and reactive groups that may be utilized for coupling a payload to the N-terminus, C-terminus or the side chain of an amino acid or amino acid derivative by chemical synthesis. Typical reactions that may be used to couple a payload to an amino acid-based linker by chemical synthesis include, without limitation: peptide coupling, activated ester coupling (NHS ester, PFP ester), click reaction (CuAAC, SPAAC), Michael addition (thiol maleimide conjugation). The coupling of payloads to peptides has been extensively described in the prior art, for example by Costoplus et al. (Peptide-Cleavable Self-immolative Maytansinoid Antibody-Drug Conjugates Designed To Provide Improved Bystander Killing. ACS Med Chem Lett. 2019 Sep. 27; 10(10):1393-1399), Sonzini et al. (Improved Physical Stability of an Antibody-Drug Conjugate Using Host-Guest Chemistry. Bioconjug Chem. 2020 Jan. 15; 31(1):123-129), Bodero et al. (Synthesis and biological evaluation of RGD and isoDGR peptidomimetic-α-amanitin conjugates for tumor-targeting. Beilstein J. Org. Chem. 2018, 14, 407-415), Nunes et al. (Use of a next generation maleimide in combination with THIOMAB™ antibody technology delivers a highly stable, potent and near homogeneous THIOMAB™ antibody-drug conjugate (TDC). RSC Adv., 2017, 7, 24828-24832), Doronina et al. (Enhanced activity of monomethylauristatin F through monoclonal antibody delivery: effects of linker technology on efficacy and toxicity. Bioconjug Chem. 2006 Jan-Feb; 17(1):114-24), Nakada et al. (Novel antibody drug conjugates containing exatecan derivative-based cytotoxic payloads. Bioorg Med Chem Lett. 2016 Mar. 15; 26(6):1542-1545) and Dickgiesser et al. (Site-Specific Conjugation of Native Antibodies Using Engineered Microbial Transglutaminases. Bioconjug Chem. 2020 Mar 12. doi: 10.1021/acs.bioconjchem.0c00061).

It is to be understood that the payload may be coupled to the N-terminal or to the C-terminal end of a peptide-based or a peptide-comprising linker according to the invention. In certain embodiments, a payload may be coupled directly to the N-terminal amino group or the C-terminal carboxyl group of a peptide or an amino acid residue.

The skilled person is aware of reactive groups that are suitable for coupling a payload to an amino acid residue. For example, an amine-comprising payload may be coupled to the C-terminal carboxyl group of an amino acid residue via an amide bond. Alternatively, a payload comprising a thiol group or and hydroxyl group may be coupled to the C-terminal carboxyl group of an amino acid via a thioester or an ester bond, respectively. A payload comprising a carboxylic acid group may be coupled to the N-terminal amino group of an amino acid residue via an amide bond.

In certain embodiments, a payload may be coupled indirectly to the N- or C-terminal end of a peptide or amino acid residue comprised in the linker according to the invention. The skilled person is aware of linker molecules that may be used to couple a payload to the N-terminal amino group or the C-terminal carboxyl group of an amino acid residue comprised in the linker according to the invention.

In certain embodiments, a payload comprising a hydroxyl group may be coupled to the N-terminus of an amino acid residue via a linker molecule. For example, payloads comprising a hydroxyl group may be coupled to an N-terminal amino group via a carbamate linker molecule. In certain embodiments, a payload comprising a thiol group may be coupled to the N-terminus of an amino acid residue via a linker molecule. For example, payloads comprising a thiol group may be coupled to an N-terminal amino group via a thiocarbamate linker molecule.

Alternatively, payloads comprising a thiol group may be coupled to an N-terminal amino group via an alkyl linker molecule comprising a carboxyl group and a thiol group. In certain embodiments the alkyl linker molecule may be a 3-mercaptopropionic acid linker molecule, wherein the payload forms a di-sulfur bond with the thiol group comprised in the 3-mercaptopropionic acid linker molecule.

In certain embodiments, a payload comprising an amide group may be coupled to the N-terminus of an amino acid residue via a linker molecule. For example, payloads comprising an amine group may be coupled to an N-terminal amino group via a dicarboxylic acid linker molecule, wherein the dicarboxylic acid linker forms an amide bond with the payload and the amino group of the N-terminal amino acid residue. Examples of dicarboxylic acids that may be used as linker molecules in the present invention are, without limitation, succinic acid or pimelic acid.

Alternative linker molecules for indirectly coupling payloads to the N-terminus of an amino acid residue comprised in the linker according to the invention or linker molecules that are suitable for indirectly coupling payloads to the C-terminus of an amino acid residue comprised in the linker according to the invention have been described in the art and are encompassed by the present invention.

In a particular embodiment, the invention relates to the method according to the invention, wherein the payload comprises at least one of:
a toxin;
a cytokine;
a growth factor;
a radionuclide;
a hormone;
an anti-viral agent;
an anti-bacterial agent;
a fluorescent dye;
an immunoregulatory/immunostimulatory agent;
a half-life increasing moiety;
a solubility increasing moiety;
a polymer-toxin conjugate;
a nucleic acid;
a biotin or streptavidin moiety;
a vitamin;
a protein degradation agent (‘PROTAC’);
a target binding moiety; and/or
an anti-inflammatory agent.

Any one of the payloads disclosed herein may either be directly coupled to a linker for use in the one-step conjugation process disclosed herein or may be linked to a linking moiety comprised in an antibody-linker conjugate that has been generated as part of the two-step process disclosed herein.

In certain embodiments, the payload may be a cytokine. The term “cytokine,” as used herein, means any secreted polypeptide that affects the functions of other cells, and that modulates interactions between cells in the immune or inflammatory response. Cytokines include, but are not limited to monokines, lymphokines, and chemokines regardless of which cells produce them. For instance, a monokine is generally referred to as being produced and secreted by a monocyte, however, many other cells produce monokines, such as natural killer cells, fibroblasts, basophils, neutrophils, endothelial cells, brain astrocytes, bone marrow stromal cells, epidermal keratinocytes, and B-lymphocytes. Lymphokines are generally referred to as being produced by lymphocyte cells. Examples of cytokines include, but are not limited to, interleukin-1 (IL-1), interleukin-6 (IL-6), Tumor Necrosis Factor alpha (TNFα), and Tumor Necrosis Factor beta (TNFβ).

In certain embodiments, the payload may be an anti-inflammatory agent. As used herein, the term “anti-inflammatory agent” means those agent classes whose main mode of action and use is in the area of treating inflammation and also any other agent from another therapeutic class that possesses useful anti-inflammatory effects. Such anti-inflammatory agents include, but are not limited to non-steroidal anti-inflammatory drugs (NSAIDs), disease modifying anti-rheumatic drugs (DMARDs), macrolide antibiotics and statins. Preferably, the NSAIDs include, but are not limited to, salicylates (e.g. aspirin), arylpropionic acids (e.g. ibuprofen), anthranilic acids (e.g. mefenamic acid), pyrazoles (e.g. phenylbutazone), cyclic acetic acids (indomethicin) and oxicams (e.g. piroxicam). Preferably, anti-inflammatory agents for use in the methods of the present invention include sulindac, diclofenac, tenoxicam, ketorolac, naproxen, nabumetone, diflunasal, ketoprofen, arlypropionic acids, tenidap, hydroxychloroquine, sulfasalazine, celecoxib, rofecoxib, meloxicam, etoricoxib, valdecoxib, methotrexate, etanercept, infliximab, adalimumab, atorvastatin, fluvastatin, lovastatin, pravastatin, simvastatin, clarithromycin, azithromycin, roxithromycin, erythromycin, ibuprofen, dexibuprofen, flurbiprofen, fenoprofen, fenbufen, benoxaprofen, dexketoprofen, tolfenamic acid, nimesulide and oxaprozin.

In certain embodiments, the anti-inflammatory agent may be an anti-inflammatory cytokine, which, when conjugated to a target specific antibody, can ameliorate inflammations caused, e.g., by autoimmune diseases. Cytokines with anti-inflammatory activities may be, without limitation, IL-1RA, IL-4, IL-6, IL-10, IL-11, IL-13 or TGF-β.

In certain embodiments, the payload may be a growth factor. The term “growth factor” as used herein refers to a naturally occurring substance capable of stimulating cellular growth, proliferation, cellular differentiation, and/or cellular maturation. Growth factors exist in the form of either proteins or steroid hormones. Growth factors are important for regulating a variety of cellular processes. Growth factors typically act as signaling molecules between cells. However, their ability to promote cellular growth, proliferation, cellular differentiation, and cellular maturation varies between growth factors. A non-limiting list of examples of growth factors includes: basic fibroblast growth factor, adrenomedullin, angiopoietin, autocrine motility factor, bone morphogenetic proteins, brain-derived neurotrophic factor, epidermal growth factor, epithelial growth factor, fibroblast growth factor, glial cell line-derived neurotrophic factor, granulocyte colony-stimulating factor, granulocyte macrophage colony-stimulating factor, growth differentiation factor-9, hepatocyte growth factor, hepatoma-derived growth factor, insulin growth factor, insulin-like growth factor, migration-stimulating factor, myostatin, nerve growth factor, and other neurotrophins, platelet-derived growth factor, transforming growth factor alpha, transforming growth factor beta, tumor-necrosis-factor-alpha, vascular endothelial growth factor, placental growth factor, fetal bovine somatotrophin, and cytokines (e.g. IL-1-cofactor for IL-3 and IL-6, IL-2-t-cell growth factor, IL-3, IL-4, IL-5, IL-6, and IL-7).

In certain embodiments, the payload may be a hormone. The term “hormone”, as used herein, refers to a chemical released by a cell or a gland in one part of the body that sends out messages that affect cells in other parts of the organism. Examples of hormones that are useful in the present invention are, without limitation, melatonin (MT), serotonin (5-HT), thyroxine (T4), triiodothyronine (T3), epinephrine or adrenaline (EPI), norepinephrine or noradrenaline (NRE), dopamine (DPM or DA), antimullerian hormone or mullerian inhibiting hormone (AMH), adiponectin (Acrp30), adrenocorticotropic hormone or corticotrophin (ACTH), angiotensinogen and angiotensin (AGT), antidiuretic hormone or vasopressin (ADH), atrial natriuretic peptide or atriopeptin (ANP), calcitonin (CT), cholecystokinin (CCK), corticotrophin-releasing hormone (CRH), erythropoietin (EPO), follicle-stimulating hormone (FSH), gastrin (GRP), ghrelin, glucagon (GCG), gonadotrophin-releasing hormone (GnRH), growth hormone-releasing hormone (GHRH), human chorionic gonadotrophin (hCG), human placental lactogen (HPL), growth hormone (GH or hGH), inhibin, insulin (INS), insulin-like growth factor or somatomedin (IGF), leptin (LEP), luteinizing hormone (LH), melanocyte stimulating hormone (MSH or α-MSH), orexin, oxytocin (OXT), parathyroid hormone (PTH), prolactin (PRL), relaxin (RLN), secretin (SCT), somatostatin (SRIF), thrombopoietin (TPO), thyroid-stimulating hormone or thyrotropin (TSH), thyrotropin-releasing hormone (TRH), cortisol, aldosterone, testosterone, dehydroepiandrosterone (DHEA), androstenedione, dihydrotestosterone (DHT), estrone, estriol (E3), progesterone, calcitriol, calcidiol, prostaglandins (PG), leukotrienes (LT), prostacyclin (PGI2), thromboxane (TXA2), prolactin releasing hormone (PRH), lipotropin (PRH), brain natriuretic peptide (BNP), neuropeptide Y (NPY), histamine, endothelin, pancreatic polypeptide, renin and enkephalin.

In certain embodiments, the payload may be an antiviral agent. The term “antiviral agent” as used herein means an agent (compound or biological) that is effective to inhibit the formation and/or replication of a virus in a mammal. This includes agents that interfere with either host or viral mechanisms necessary for the formation and/or replication of a virus in a mammal. Antiviral agents include, for example, ribavirin, amantadine, VX-497 (merimepodib, Vertex Pharmaceuticals), VX—498 (Vertex Pharmaceuticals), Levovirin, Viramidine, Ceplene (maxamine), XTL-001 and XTL-002 (XTL Biopharmaceuticals).

In certain embodiments, the payload may be an antibacterial agent. The term “antibacterial agent” as used herein refers to any substance, compound, a combination of substances, or a combination of compounds capable of: (i) inhibiting, reducing or preventing growth of bacteria; (ii) inhibiting or reducing ability of a bacteria to produce infection in a subject; or (iii) inhibiting or reducing ability of bacteria to multiply or remain infective in the environment. The term “antibacterial agent” also refers to compounds capable of decreasing infectivity or virulence of bacteria.

In certain embodiments, the payload may be an immunoregulatory agent. The term “immunoregulatory agent” as used herein for combination therapy refers to substances that act to suppress, mask, or enhance the immune system of the host. Examples of immunomodulatory agents include, but are not limited to, proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules, iRNA and triple helices), small molecules, organic compounds, and inorganic compounds. In particular, immunomodulatory agents include, but are not limited to, methothrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunamide), T cell receptor modulators, and cytokine receptor modulators.

In certain embodiments, the immunoregulatory agent may be an immunostimulatory agent. The term “immunostimulatory agent” as used herein preferably refers to any substance or substance that can trigger an immune response (e.g., an immune response against a particular pathogen). Immune cell activating compounds include Toll-like receptor (TLR) agonists. Such agonists include pathogen associated molecular patterns (PAMPs), e.g., an infection-mimicking composition such as a bacterially-derived immunomodulator (a.k.a., danger signal) and damage associated molecular pattern (DAMPs), e.g. a composition mimicking a stressed or damaged cell. TLR agonists include nucleic acid or lipid compositions (e.g., monophosphoryl lipid A (MPLA)). In one example, the TLR agonist comprises a TLR9 agonist such as a cytosine-guanosine oligonucleotide (CpG-ODN), a poly(ethylenimine) (PEI)-condensed oligonucleotide (ODN) such as PEI-CpG-ODN, or double stranded deoxyribonucleic acid (DNA). In another example, the TLR agonist comprises a TLR3 agonist such as polyinosine-polycytidylic acid (poly (I:C)), PEI-poly (I:C), polyadenylic-polyuridylic acid (poly (A:U)), PEI-poly (A:U), or double stranded ribonucleic acid (RNA). Other exemplary vaccine immunostimulatory compounds include lipopolysaccharide (LPS), chemokines/cytokines, fungal beta-glucans (such as lentinan), imiquimod, CRX-527, and OM-174.

In certain embodiments, the payload may be a half-life increasing moiety or a solubility increasing moiety. Half-life increasing moieties are, for example, PEG-moieties (polyethylenglycol moieties; PEGylation), other polymer moieties, PAS moieties (oliogopeptides comporising Proline, Alanine and Serine; PASylation), or Serum albumin binders. Solubility increasing moieties are, for example PEG-moieties (PEGylation) or PAS moieties (PASylation).

In certain embodiments, the payload may be a polymer-toxin conjugate. Polymer-toxin conjugates are polymers that are capable of carrying many payload molecules. Such conjugates are sometimes also called fleximers, as e.g. marketed by Mersana therapeutics. A polymer-toxin conjugate may comprise any of the toxins disclosed herein.

In certain embodiments, the payload may be a nucleotide. One example of a nucleic acid payload is MCT-485, which is a very small non-coding double stranded RNA which has oncolytic and immune activating properties, developed by MultiCell Technologies, Inc.

In certain embodiments, the payload may be a fluorescent dye. The term “fluorescent dye” as used herein refers to a dye that absorbs light at a first wavelength and emits at second wavelength that is longer than the first wavelength. In certain embodiment, the fluorescent dye is a near-infrared fluorescent dye, which emits light at a wavelength between 650 and 900 nm. In this region, tissue autofluorescence is lower, and less fluorescence extinction enhances deep tissue penetration with minimal background interference. Accordingly, near-infrared fluorescent imaging may be used to make tissues that are bound by the antibody-payload conjugate of the invention visible during surgery. “Near-infrared fluorescent dyes” are known in the art and commercially available. In certain embodiments, the near-infrared fluorescent dye may be IRDye 800CW, Cy7, Cy7.5, NIR CF750/770/790, DyLight 800 or Alexa Fluor 750.

In certain embodiments, the payload may comprise a radionuclide. The term “radionuclide”, as used herein, relates to medically useful radionuclides, including, for example, positively charged ions of radiometals such as Y, In, Tb, Ac, Cu, Lu, Tc, Re, Co, Fe and the like, such as ⁹⁰Y, ¹¹¹In, ⁶⁷Cu, ⁷⁷Lu, ⁹⁹Tc, ¹⁶¹Tb, ²²⁵Ac and the like. The radionuclide may be comprised in a chelating agent such as DOTA or NODA-GA. Further, the radionuclide may be a therapeutic radionuclide or a radionuclide that can be used as contrast agent in imaging techniques as discussed below. Radionuclides or molecules comprising radionuclides are known in the art and commercially available.

In certain embodiments, the payload may be a vitamin. The vitamin may be selected from the group consisting of folates, including folic acid, folacin, and vitamin B9.

In a particular embodiment, the invention relates to the method according to the invention, wherein the toxin is at least one selected from the group consisting of
a pyrrolobenzodiazepine (e.g., PBD);
an auristatin (e.g., MMAE, MMAF);
a maytansinoid (e.g., maytansine, DM1, DM4, DM21);
a duocarmycin;
a nicotinamide phosphoribosyltransferase (NAMPT) inhibitor;
a tubulysin;
an enediyne (e.g., calicheamicin);
an anthracycline derivative (PNU) (e.g., doxorubicin);
a pyrrole-based kinesin spindle protein (KSP) inhibitor;
a cryptophycin;
a drug efflux pump inhibitor;
a sandramycin;
an amanitin (e.g., α-amanitin); and
a camptothecin (e.g., exatecans, deruxtecans).

That is, the antibody-linker conjugates produced with the method of the invention preferably comprise a toxin payload. The term “toxin” as used herein relates to any compound produced by living cells or organisms and poisonous to a cell or organism. Toxins, thus can be, e.g. small molecules, peptides, or proteins. Specific examples are neurotoxins, necrotoxins, hemotoxins and cytotoxins. In certain embodiments, the toxin is toxin that is used in the treatment of neoplastic diseases. That is, the toxin may be conjugated to an antibody with the method of the invention and delivered to or into a malignant cell due to the target specificity of the antibody.

In certain embodiments, the toxin may be an auristatin. As used herein, the term “auristatin” refers to a family of anti-mitotic agents. Auristatin derivatives are also included within the definition of the term “auristatin”. Examples of auristatin include, but are not limited to, synthetic analogues of auristatin E (AE), monomethyl auristatin E (MMAE), monomethyl auristatin F (MMAF) and dolastatin.

In certain embodiments, the toxin may be a maytansinoid. In the context of the present invention, the term “maytansinoid” refers to a class of highly cytotoxic drugs originally isolated from the African shrub Maytenus ovatus and further maytansinol (Maytansinol) and C-3 ester of natural maytansinol (U.S. Pat. No. 4,151,042); C-3 ester analog of synthetic maytansinol (Kupchan et al., J. Med. Chem. 21: 31-37, 1978; Higashide et al., Nature 270: 721-722, 1977; Kawai et al., Chem. Farm. Bull. 32: 3441-3451; and U.S. Pat. No. 5,416,064); C-3 esters of simple carboxylic acids (U.S. Pat. Nos. 4,248,870; 4,265,814; 4,308,268; 4,308,269; 4,309,428; 4,317,821; 4,322,348; and 4,331,598); and C-3 esters with derivatives of N-methyl-L-alanine (U.S. Pat. Nos. 4,137,230; 4,260,608; and Kawai et al., Chem. Pharm Bull. 12: 3441, 1984). Exemplary maytansinoids that may be used in the method of the invention or that may be comprised in the antibody-payload conjugate of the invention are maytansine, DM1, DM3, DM4 and/or DM21.

In certain embodiments, the toxin may be a duocarmycin. Suitable duocarmycins may be e.g. duocarmycin A, duocarmycin B1, duocarmycin B2, duocarmycin C1, duocarmycin C2, duocarmycin D, duocarmycin SA, duocarmycin MA, and CC-1065. The term “duocarmycin” should be understood as referring also to synthetic analogs of duocarmycins, such as adozelesin, bizelesin, carzelesin, KW-2189 and CBI-TMI.

In certain embodiments, the toxin may be a NAMPT inhibitor. As used herein, the terms “NAMPT inhibitor” and “nicotinamide phosphoribosyl transferase inhibitor” refer to an inhibitor that reduces the activity of NAMPT. The term “NAMPT inhibitor” may also include prodrugs of a NAMPT inhibitor. Examples of NAMPT inhibitors include, without limitation, FK866 (also referred to as AP0866), GPP 78 hydrochloride, ST 118804, STF31, pyridyl cyanoguanidine (also referred to as CH-828), GMX-1778, and P7C3. Additional NAMPT inhibitors are known in the art and may be suitable for use in the compositions and methods described herein. See, e.g., PCT Publication WO 2015/054060, U.S. Pat. Nos. 8,211,912, and 9,676,721, which are incorporated by reference herein in their entireties. In some embodiments, the NAMPT inhibitor is FK866. In some embodiments, the NAMPT inhibitor is GMX-1778.

In certain embodiments, the toxin may be a tubulysin. Tubulysins are cytotoxic peptides, which include 9 members (A-I). Tubulysin A has potential application as an anticancer agent. It arrests cells in the G2/M phase. Tubulysin A inhibits polymerization more efficiently than vinblastine and induces depolymerization of isolated microtubules. Tubulysin A has potent cytostatic effects on various tumor cell lines with IC50 in the picomolar range. Other tubulysins that may be used in the method of the invention may be tubulysin E.

In certain embodiments, the toxin may be an enediyne. The term “enediyne,” as used herein, refers to a class of bacterial natural products characterized by either nine- and ten-membered rings containing two triple bonds separated by a double bond (see, e.g., K. C. Nicolaou; A. L. Smith; E. W. Yue (1993). “Chemistry and biology of natural and designed enediynes”. PNAS 90 (13): 5881-5888; the entire contents of which are incorporated herein by reference). Some enediynes are capable of undergoing Bergman cyclization, and the resulting diradical, a 1,4-dehydrobenzene derivative, is capable of abstracting hydrogen atoms from the sugar backbone of DNA which results in DNA strand cleavage (see, e.g., S. Walker; R. Landovitz; W. D. Ding; G. A. Ellestad; D. Kahne (1992). “Cleavage behavior of calicheamicin gamma 1 and calicheamicin T”. Proc Natl Acad Sci U.S.A. 89 (10): 4608-12; the entire contents of which are incorporated herein by reference). Their reactivity with DNA confers an antibiotic character to many enediynes, and some enediynes are clinically investigated as anticancer antibiotics. Nonlimiting examples of enediynes are dynemicin, neocarzinostatin, calicheamicin, esperamicin (see, e.g., Adrian L. Smith and K. C. Bicolaou, “The Enediyne Antibiotics” J. Med. Chem., 1996, 39 (11), pp 2103-2117; and Donald Borders, “Enediyne antibiotics as antitumor agents,” Informa Healthcare; 1st edition (Nov. 23, 1994, ISBN-10: 0824789385; the entire contents of which are incorporated herein by reference). In a particular embodiment, the toxin may be calicheamicin.

In certain embodiments, the toxin may be a doxorubicin. “Doxorubicin” as used herein refers to members of the family of Anthracyclines derived from Streptomyces bacterium Streptomyces peucetius var. caesius, and includes doxorubicin, daunorubicin, epirubicin and idarubicin.

In certain embodiments, the toxin may be a kinesin spindle protein inhibitor. The term “kinesin spindle protein inhibitor” refers to a compound that inhibits the kinesin spindle protein, which involves in the assembly of the bipolar spindle during cell division. Kinesin spindle protein inhibitors are being investigated for the treatment of cancer. Examples of kinesin spindle protein inhibitor include ispinesib. Further, the term “kinesin spindle protein inhibitor” includes SB715992 or SB743921 from GlaxoSmithKline and pentamidine/chlorpromarine from CombinatoRx.

In certain embodiments, the toxin may a cryptophycin as described in US20180078656A1, which is incorporated by reference.

In certain embodiments, the toxin may be sandramycin. Sandramycin is a depsipeptide that has first been isolated from Nocardioides sp. (ATCC 39419) and has been shown to have cytotoxic and anti-tumor activity.

In certain embodiments, the toxin may be an amatoxin. Amatoxins (including alpha-amanitin, beta-amanitin and amanitin) are cyclic peptides composed of 8 amino acids. They can be isolated from Amanita phalloides mushrooms or prepared from the building blocks by synthesis. Amatoxins inhibit specifically the DNA-dependent RNA polymerase II of mammalian cells, and by this transcription and protein biosynthesis of the cells affected. Inhibition of transcription in a cell causes stop of growth and proliferation. Though not covalently bound, the complex between amanitin and RNA-polymerase II is very tight (KD=3 nM). Dissociation of amanitin from the enzyme is a very slow process what makes recovery of an affected cell unlikely. When in a cell the inhibition of transcription will last too long, the cell undergoes programmed cell death (apoptosis). In one preferred embodiment, term “Amatoxin” as used herein refers to an alpha-amanitin or variant thereof as described e.g. in WO2010/115630, WO2010/115629, WO2012/119787, WO2012/041504, and WO2014/135282.

In certain embodiments, the toxin may be a camptothecin. The term “camptothecin” as used herein is intended to mean a camptothecin or camptothecin derivative that functions as a topoisomerase I inhibitor. Exemplary camptothecins include, for example, topotecan, exatecan, deruxtecan, irinotecan, DX-8951f, SN38, BN 80915, lurtotecan, 9-nitrocamptothecin and aminocamptothesin. A variety of camptothecins have been described, including camptothecins used to treat human cancer patients. Several camptothecins are described, for example, in Kehrer et al., Anticancer Drugs, 12 (2): 89-105, (2001) or Li et al., ACS Med. Chem. Lett. 2019, 10, 10, 1386-1392).

The toxin, in the sense of the present invention may also be an inhibitor of a drug efflux transporter. Antibody-payload conjugates comprising a toxin and an inhibitor of a drug efflux transporter may have the advantage that, when internalized into a cell, the inhibitor of the drug efflux transporter prevents efflux of the toxin out of the cell. Within the present invention, the drug efflux transporter may be P-glycoprotein. Some common pharmacological inhibitors of P-glycoprotein include: amiodarone, clarithromycin, ciclosporin, colchicine, diltiazem, erythromycin, felodipine, ketoconazole, lansoprazole, omeprazole and other proton-pump inhibitors, nifedipine, paroxetine, reserpine, saquinavir, sertraline, quinidine, tamoxifen, verapamil, and duloxetine. Elacridar and CP 100356 are other common P-gp inhibitors. Zosuquidar and tariquidar were also developed with this in mind. Lastly, valspodar and reversan are other examples of such agents.

It is to be understood that the payload B as defined herein is not exclusively to be understood as the actual payload as such but rather as a payload molecule. A payload molecule, as used herein, may comprise additional structures, for example to facilitate coupling of a payload to a linking moiety B or to the K residue or the chemical spacers via chemical synthesis.

That is, in certain embodiments, the actual payload may be comprised in a payload molecule that is linked to the linker of the invention. A payload molecule may have the structure:
X-(spacer)-payload,
wherein payload represents the actual payload, e.g., one of the compounds disclosed herein, X represents a reactive group that is suitable for attaching the payload molecule to a compatible functional group in a linking moiety (two-step process) or in a chemical spacer or K residue of a linker (one-step process), and wherein (spacer) represents a chemical spacer that spatially separates the actual payload from the reactive group X. However, it is to be understood that in certain embodiments, the reactive group X may be part of the spacer or the actual payload. For example, the spacer may comprise a peptide or an amino acid residue, wherein the reactive group X may be the amino group of the N-terminal amino acid residue comprised in the spacer. In other embodiments, the spacer may be absent. In embodiments, where the spacer is absent, the functional group may be comprised in the actual payload. In certain embodiments, a spacer may be used to attach a functional group of interest, i.e., a functional group that is compatible with a functional group comprised in a linking moiety, to the actual payload. In certain embodiments, the reactive group X may be a maleimide group or a cyclooctyne group such as, without limitation, a DBCO or BCN group.

In a particular embodiment, the invention relates to the method according to the invention, wherein the chemical spacer (Sp₂) comprises a self-immolative moiety.

That is, the linker may comprise a self-immolative moiety to facilitate the release of the payload in the target cell or tissue. The self-immolative moiety may be comprised in any part of the linker. However, the self-immolative moiety is preferably comprised in the chemical spacer (Sp₂) which separates the payload from the K residue. Alternatively, the self-immolative moiety may be comprised in the (spacer) that is comprised in the payload molecule as defined above.

As used herein, the term “self-immolative moiety” refers to an at least bifunctional molecule that can be included in a linker and degrades spontaneously after an initial reaction has taken place and thereby releases the payload. The initial reaction may be the hydrolysis of a covalent bond between the self-immolative moiety and an amino acid residue. In certain embodiments, the covalent bond between the self-immolative moiety and an amino acid residue may be an amide bond formed between the α-carboxyl group of the amino acid and an amine group comprised in the self-immolative moiety and the initial reaction may be catalyzed by a peptidase or a protease. However, other chemistries are encompassed by this invention.

In a particular embodiment, the invention relates to the method according to the invention, wherein the self-immolative moiety is directly attached to the payload B.

More preferably, the self-immolative moiety is directly attached to the payload B, such that the payload is released upon degradation of the self-immolative moiety. In certain embodiments, the self-immolative moiety is located between the payload and the K residue comprised in the linker. That is, the self-immolative moiety may be coupled to the N-terminus of the K residue or to the C-terminus of the K residue. Alternatively, the self-immolative moiety may be located between the payload and an amino acid residue comprised in the chemical spacer (Sp₂), preferably at the N- or C-terminus of said amino acid residue. Further, the self-immolative moiety may be located between the payload and a non-amino acid residue comprised in the chemical spacer (Sp₂) by any method known in the art.

It is to be understood that the choice of the self-immolative moiety depends, amongst others on the functional groups that are available in a payload molecule.

In a particular embodiment, the invention relates to the method according to the invention, wherein the self-immolative moiety comprises a p-aminobenzyl carbamoyl (PABC) moiety or an amino methylene spacer.

That is, in certain embodiments, a linker may comprise the self-immolative moiety p-aminobenzyl carbamoyl (PABC). PABC comprises a free amine group which is suitable for coupling to the C-terminus of an amino acid residue or peptide and a carbamoyl group via which it can be coupled to a payload, in particular an amine-comprising payload. However, the skilled person is aware of methods to functionalize a payload such that it comprises an amine group. The self-immolative moiety PABC is preferably located between the payload and an amino acid residue comprised in the linker. The amino acid residue is preferably the residue K or an amino acid comprised in the chemical spacer (Sp₂). In certain embodiments, the self-immolative moiety PABC is located between the payload and an alanine residue comprised in the chemical spacer (Sp₂). In certain embodiments, the self-immolative moiety may be located between a payload and a peptidase cleavage site. In certain embodiments, the self-immolative moiety may be located between a payload and a cathepsin cleavage site. That is, the self-immolative moiety may be located between a payload and a motif that is known to be cleavable by a cathepsin.

The term “cathepsin”, as used herein, refers to a family of proteases. The term cathepsin comprises cathepsin A, cathepsin B, cathepsin C, cathepsin D, cathepsin E, cathepsin F, cathepsin G, cathepsin H, cathepsin K, cathepsin L1, cathepsin L2, cathepsin O, cathepsin S, cathepsin W and cathepsin Z. In a particular embodiment, the cleavable moiety may be a motif that is specifically hydrolyzed by cathepsin B, such as valine-alanine, valine-citrulline or alanine-alanine. Further motifs that can be specifically hydrolyzed by a peptidase have been disclosed by Salomon et al., Optimizing Lysosomal Activation of Antibody-Drug Conjugates (ADCs) by Incorporation of Novel Cleavable Dipeptide Linkers, Mol Pharm. 2019, 16(12), p.4817-4825.

One typical dipeptide structure used in ADC linkers is the valine-citrulline motif, as e.g. provided in Brentuximab Vedotin, and discussed in Dubowchik and Firestone; Cathepsin B-labile dipeptide linkers for lysosomal release of doxorubicin from internalizing immunoconjugates: model studies of enzymatic drug release and antigen-specific in vitro anticancer activity; Bioconjug Chem; 2002; 13(4); p.855-69. This linker can be cleaved by cathepsin B to release the actual payload at the site of disease. The same applies to the valine-alanine motif, which is for example provided in SGN-CD33A.

Thus, in certain embodiments, the linker may comprise the structure (Sp₁)-K-(Sp₂)-Val-Cit-(self-immolative moiety)-Payload. In certain embodiments, the linker may comprise the structure (Sp₁)-K-(Sp₂)-Val-Cit-Payload. In certain embodiments, the linker may comprise the structure (Sp₁)-K-(Sp₂)-Val-Cit-PABC-Payload.

In certain embodiments, the linker may comprise or consist of the structure K-Val-Cit-(self-immolative moiety)-Payload. In certain embodiments, the linker may comprise or consist of the structure K-Val-Cit-PABC-Payload. In certain embodiments, the linker may comprise or consist of the structure K-Val-Cit-PABC-MMAE. In certain embodiments, the linker may comprise or consist of the structure K-Val-Cit-PABC-maytansine.

It has to be noted that the peptide cleavage site may also be a motif that is cleavable by other peptidases such as Caspase 3, Legumain or Neutrophil elastase or as described by Dal Corso et al., Innovative Linker Strategies for Tumor-Targeted Drug Conjugates; Chemistry; 25(65); p.14740-14757.

However, it has to be noted that cells comprise a wide range of cellular peptidases and that also other, less conserved amino acid motifs may be efficiently cleaved by a peptidase. Thus, in certain embodiments, the linker may comprise the structure (Sp₁)-K-(Sp₂)-PABC-Payload, wherein (Sp₂) is absent or consists of amino acid residues.

In certain embodiments, the linker may comprise the structure (Sp₁)-K-(Sp₂)-PABC-Payload, wherein (Sp₂) comprises a PEG moiety between the PABC moiety and the most C-terminal amino acid residue comprised in (Sp₂) or in the K residue.

In certain embodiments, linkers comprising the self-immolative moiety PABC are coupled to an amine-comprising payload, in particular payloads comprising a primary or a secondary amine. In certain embodiments, the amine-comprising payload is an ausristatin, such as MMAE. In certain embodiments, the amine-comprising payload is a maytansinoid, such as maytansine.

It has to be noted that the payload may be coupled to self-immolative PABC moiety via an additional linker molecule. For example, an amine-comprising payload may be coupled to the PABC moiety via a p-nitrophenol (PNP) group. Further linker molecules that allow coupling of payloads comprising other reactive groups than amine to a PABC moiety have been disclosed by Su et al., Bioconjugate Chem. 2018, 29, 4, 1155-1167; and Dokter et al., Mol Cancer Ther. 2014 Nov; 13(11):2618-29. For example, payloads comprising an alcohol or phenol group may be coupled to PABC via an ethylene diamine (EDA) linker.

In a particular embodiment, the invention relates to the method according to the invention, wherein the self-immolative moiety comprises a methyl amine group. It has been demonstrated previously that methyl amine groups can be used as self-immolative moieties in peptide based linkers of ADCs (Costoplus et al., ACS Med. Chem. Lett., 2019, 10, 10, 1393-1399 and Li et al., ACS Med. Chem. Lett. 2019, 10, 10, 1386-1392).

In particular, the self-immolative moiety comprising a methyl amine group may be coupled to the C-terminal end of an amino acid residue via an amide bond formed between the α-carboxyl group of the amino acid residue and the amine comprised in the methyl amine group. The amino acid residue may be an amino acid residue comprised in (Sp₂) or the residue K. The methyl group comprised in the methyl amine group may be coupled to the payload by an ether or thioether bond. Thus, a methyl amine group may be preferably used as a self-immolative group when the payload comprises a hydroxyl or thiol group. In certain embodiments, the hydroxyl-comprising payload may be camptothecin, such as the exatecan derivative Dxd or an anthracycline, such as PNU-159682. In certain embodiments the thiol-comprising payload may be a maytansinoid such as DM1, DM4 or DM21.

A linker comprising an amino methylene spacer may comprise the molecular structure C—(NH)—(CH₃)—O—C or C—(NH)—(CH₃)—S—C.

It is to be understood that PABC and self-immolative moieties comprising an amino methylene spacer are preferably used to couple payloads to the C-terminal carboxyl group of an amino acid residue.

Other self-immolative moieties that may be used to couple payloads to the C-terminal carboxyl group of an amino acid residue comprise p-aminobenzyl ethanol (PABE) linkers for the coupling of phenol-comprising payloads to the C-terminal carboxyl group of an amino acid residue (Zhang et al., Bioconjugate Chem. 2018, 29, 6, 1852-1858) or para-methyl aniline (PMA) linkers for the coupling of payloads comprising a tertiary amine or a heteroaryl moiety to the C-terminal carboxyl group of an amino acid residue (Staben et al., Nature Chemistry volume 8, pages 1112-1119(2016)). Non-limiting examples of payloads comprising a phenol group are duocarmycin GA or the pyrrolobenzodiazepine PBD. A non-limiting example of a payload comprising a tertiary amine is duocarmycin GA.

However, payloads may also be coupled to the N-terminal amino group via a self-immolative moiety. For example, payloads may be coupled to the N-terminal amino group of an amino acid residue via a self-immolative moiety comprising an ortho-hydroxy-protected aryl sulfate. For example, an ortho-hydroxy-protected aryl sulfate (OHPAS) may be used to couple a phenolic payload, such as PBD, to the N-terminal amino group of an amino acid residue. The OHPAS moiety preferably comprises a carboxyl group via which it can be coupled directly to the N-terminal amino group of an amino acid residue. Alternatively, the OHPAS moiety may be coupled to the N-terminal amino group of an amino acid residue via a functionalized PEG linker, for example, without limitation, a functionalized (PEG)₂ linker. Preferably, the PEG linker is functionalized on one end with an amino group to allow coupling to the carboxyl group comprised in the OHPAS moiety and with a carboxyl group on the other side to allow coupling to the N-terminal amino group of an amino acid residue (Park et al., Bioconjugate Chem. 2019, 30, 7, 1957-1968).

Alternatively or in addition, a linker molecule may be located between the sulfate group of OHPAS and the payload to allow coupling of non-phenolic payloads to OHPAS. For example, a para-hydroxy benzyl (PHB) linker molecule may be used to allow coupling of payloads comprising a primary or secondary amine to an OHPAS moiety through the formation of a carbamate. Payloads comprising a tertiary amine may be coupled to an OHPAS comprising linker through the formation of a quaternary ammonium. Further, a para-hydroxy benzyl ethylenediamine (PHB-EDA) linker molecule may be used to couple a hydroxyl-comprising payload to an OHPAS moiety through the formation of a carbamate (Park et al., Bioconjugate Chem. 2019, 30, 7, 1957-1968).

In certain embodiments, the payload may be coupled to an amino acid residue comprised in the linker via a cleavable moiety. A “cleavable moiety”, as used herein, is a chemical unit that can be separated from the actual payload by enzymatic or non-enzymatic hydrolysis. In certain embodiments, a cleavable moiety may be an amino acid motif that is hydrolyzable by a peptidase or protease.

In other embodiments, the cleavable moiety comprised in the linker may be a carbohydrate moiety. In such embodiments, the cleavable moiety may be a moiety that is cleavable by a glucosidase. Thus, in certain embodiments, the cleavable moiety may be a moiety that is cleavable by a beta-glucuronidase or a beta-galactosidase.

In other embodiments, the cleavable moiety comprised in the linker may be a phosphate moiety. In such embodiments, the cleavable moiety may be a moiety that is cleavable by a phosphatase. Thus, in certain embodiments, the cleavable moiety may be a moiety that is cleavable by a beta lysosomal acid pyrophosphatase or an acid phosphatase.

Examples for further cleavable moieties that may be used for the release of payloads from a linker molecule have been described by Bargh et al., Cleavable linkers in antibody-drug conjugates; Chem Soc Rev. 2019 Aug. 12; 48(16):4361-4374. In certain embodiments, the linker may comprise the structure (cleavable moiety)-(self-immolative moiety)-payload. In such an embodiment, the self-immolative moiety may degrade upon cleavage of the cleavable moiety and release the payload.

In certain embodiments, the linker may comprise a single linking moiety or payload.

In certain embodiments, the linker may comprise two or more linking moieties and/or payloads B. That is, in certain embodiments, the linker may comprise the structure
a) (Sp₁)-K-(Sp₂)-B₁-(Sp₃)-B₂-(Sp₄),
b) (Sp₄)-B₂-(Sp₁)-K-(Sp₂)-B₁-(Sp₃),
c) (Sp₁)-B₁-(Sp₂)-K-(Sp₃)-B₂-(Sp₄), or
d) (Sp₄)-B₂-(Sp₁)-B₁-(Sp₂)-K-(Sp₃).

In such embodiments, the chemical spacers (Sp₁), (Sp₂), (Sp₃) and the K residue may have the same characteristics as defined above. Further, the moieties B₁ and B₂ may be any one of the linking moieties and/or payloads defined above. Further, the chemical spacer (Sp₄) may have the same characteristics as the chemical spacers (Sp₁), (Sp₂) or (Sp₃) or may be absent.

Thus, in a particular embodiment, the invention relates to the method according to the invention, wherein the linker comprises a second linking moiety or payload B₂, in particular wherein B₂ is connected to the linker via the chemical spacer (Sp₁) or (Sp₃).

That is, the payload or linking moiety B₂ may be connected to the chemical spacer (Sp₁) or (Sp₃) or directly to the payload or linking moiety B₁. The payload or linking moiety B₂ may comprise any functional group that is suitable for coupling B₂ to a functional group comprised in (Sp₁), (Sp₃) or B₁.

In certain embodiments, the payload or linking moiety B₂ may comprise an amino group with which B₂ is connected to (Sp₃) or B₁. That is, B₂ may be connected to a carboxyl group comprised in (Sp₃) or B₁ via said amino group. In certain embodiments, the carboxyl group comprised in (Sp₃) may be a carboxyl group comprised in the C-terminal amino acid residue of the chemical spacer (Sp₃). In certain embodiments, the carboxyl group comprised in B₁ may be the α-carboxyl group of an amino acid-based payload or linking moiety. In certain embodiments, B₂ may be coupled to a carboxyl group comprised in (Sp₃) or B₁ via a linker molecule. In certain embodiments, the linker molecule may comprise a self-immolative moiety.

In certain embodiments, the payload or linking moiety B₂ may comprise a carboxyl group with which B₂ is connected to (Sp₁) or B₁. That is, B₂ may be connected to an amine group comprised in (Sp₁) or B₁ via said carboxyl group. In certain embodiments, the amine group comprised in (Sp₁) may be an amine group comprised in the N-terminal amino acid residue of the chemical spacer (Sp₁). In certain embodiments, the amine group comprised in B₁ may be the α-amino group of an amino acid-based payload or linking moiety. In certain embodiments, B₂ may be coupled to an amine group comprised in (Sp₁) or B₁ via a linker molecule. In certain embodiments, the linker molecule may comprise a self-immolative moiety.

However, it has to be noted that B₂ may also comprise other functional groups than an amine or a carboxyl group. In such embodiments, B₂ may be coupled to (Sp₁), (Sp₃) or B₁ by any method known in the art, either directly, or via a linker or self-immolative group.

In certain embodiments, the payload or linking moiety B₂ may be coupled to an amino acid side chain comprised in (Sp₁) or (Sp₃). That is, B₂ may be connected to a functional group of an amino acid side chain comprised in (Sp₁) or (Sp₃) via a compatible functional group.

In certain embodiments, (Sp₁), (Sp₂), (Sp₃) and the K residue consist exclusively of amino acids, amino acid mimetics and/or amino acid derivatives. In certain embodiments, also B₁ and/or B₂ comprise an amino acid backbone. In such embodiments, the linker may be a linear peptide or peptidomimetic. In embodiments where B₁ is an amino acid, an amino acid mimetic or an amino acid derivative, the linker may have the structure (Sp₁)-K-(Sp₂)-B₁, wherein (Sp₁)-K-(Sp₂)-B₁ is a linear peptide or peptidomimetic. In embodiments where B₁ is an amino acid, an amino acid mimetic or an amino acid derivative, the linker may have the structure (Sp₁)-K-(Sp₂)-B₁-(Sp₃), wherein (Sp₁)-K-(Sp₂)-B₁-(Sp₃) is a linear peptide or peptidomimetic. In embodiments where B₁ is an amino acid, an amino acid mimetic or an amino acid derivative, the linker may have the structure K-(Sp₂)-B₁-(Sp₃), wherein K-(Sp₂)-B₁-(Sp₃) is a linear peptide or peptidomimetic. In embodiments where B₁ is an amino acid, an amino acid mimetic or an amino acid derivative, the linker may have the structure K-(Sp₂)-B₁, wherein K-(Sp₂)-B₁ is a linear peptide or peptidomimetic. In embodiments where B₁ is an amino acid, an amino acid mimetic or an amino acid derivative, the linker may have the structure K—B₁-(Sp₃), wherein K—B₁-(Sp₃) is a linear peptide or peptidomimetic. In embodiments where B₁ is an amino acid, an amino acid mimetic or an amino acid derivative, the linker may have the structure K—B₁, wherein K—B₁ is a linear peptide or peptidomimetic.

In embodiments where B₁ and B₂ are amino acids, amino acid mimetics or amino acid derivatives, the linker may have the structure (Sp₁)-K-(Sp₂)-B₁-(Sp₃)-B₂-(Sp₄), wherein (Sp₁)-K-(Sp₂)-B₁-(Sp₃)-B₂-(Sp₄) is a linear peptide or peptidomimetic. In other embodiments where B₁ and B₂ are amino acids, amino acid mimetics or amino acid derivatives, the linker may have the structure (Sp₄)-B₂-(Sp₁)-K-(Sp₂)-B₁-(Sp₃), wherein (Sp₄)-B₂-(Sp₁)-K-(Sp₂)-B₁-(Sp₃) is a linear peptide or peptidomimetic. In other embodiments where B₁ and B₂ are amino acids, amino acid mimetics or amino acid derivatives, the linker may have the structure (Sp₄)-B₂-(Sp₁)-B₁-(Sp₂)-K-(Sp₃), wherein (Sp₄)-B₂-(Sp₁)-B₁-(Sp₂)-K-(Sp₃) is a linear peptide or peptidomimetic.

In embodiments where B₁ is not an amino acid, an amino acid mimetic or an amino acid derivative, the linker may have the structure (Sp₁)-K-(Sp₂)-B₁-(Sp₃), wherein (Sp₁)-K-(Sp₂) is a linear peptide or peptidomimetic and B₁ is connected to the C-terminal carboxyl group comprised in (Sp₂). In embodiments where B₁ is not an amino acid, an amino acid mimetic or an amino acid derivative, the linker may have the structure (Sp₁)-B₁-(Sp₂)-K-(Sp₃), wherein (Sp₂)-K-(Sp₃) is a linear peptide or peptidomimetic and B₁ is connected to the N-terminal amino group comprised in (Sp₂). However, it has to be noted that B₁ does not necessarily have to be coupled to the peptide or peptidomimetic directly. Instead, B₁ may be coupled to the peptide or peptidomimetic via a linker molecule and/or a self-immolative moiety.

In embodiments where B₁ is an amino acid, an amino acid mimetic or an amino acid derivative and B₂ is not an amino acid, an amino acid mimetic or an amino acid derivative, the linker may have the structure (Sp₁)-K-(Sp₂)-B₁-(Sp₃)-B₂-(Sp₄), (Sp₄)-B₂-(Sp₁)-K-(Sp₂)-B₁-(Sp₃), (Sp₁)-B₁- (Sp₂)-K-(Sp₃)-B₂-(Sp₄) or (Sp₄)-B₂-(Sp₁)-B₁-(Sp₂)-K-(Sp₃), wherein (Sp₁)-K-(Sp₂)-B₁-(Sp₃) or (Sp₁)-B1-(Sp₂)-K-(Sp₃) is a linear peptide or peptidomimetic and B₂ is coupled to the C-terminal carboxyl group comprised in (Sp₃), B₁ or the K residue or to the N-terminal amino group of (Sp₁), B₁ or the K residue.

In such embodiments, an antibody-payload conjugate may be generated with, for example, an antibody to payload ratio of 2 or 4, for example with one or two payloads conjugated to each Q295 residue.

In a particular embodiment, the invention relates to the method according to the invention, wherein B₁ and B₂ are identical or differ from one another.

That is, the payload or linking moieties B₁ and B₂ may be identical, i.e., have the same chemical structure, or may be structurally different. In certain embodiments, B₁ and B₂ are both payloads or are both linking moieties. In embodiments where B₁ and B₂ are both payloads, the payloads B₁ and B₂ may be identical or different payloads. In embodiments where B₁ and B₂ are both linking moieties, the linking moieties B₁ and B₂ may be identical or different linking moieties. In certain embodiments, B₁ may be a linking moiety and B₂ may be a payload or vice versa.

It is to be understood that not all payloads or linking moieties can function as intrachain payloads or linking moieties in position B₁, for example, because they do not have the functional groups to form covalent bonds with (Sp₂) or the K residue on one side, and (Sp₃), (Sp₁) or B₂ on the other side. Thus, it is preferred that in embodiments where B₁ is an intrachain payload or linking moiety, B₁ is a divalent or polyvalent molecule. For example, B₁ may be an amino acid, an amino acid mimetic or an amino acid derivative. In such embodiments, B₁ may be coupled via its amino group to the C-terminal carboxyl group of (Sp₂) or K and via its carboxyl group with the N-terminal amino group of (Sp₃) or B₂. Alternatively, B₁ may be coupled via its carboxyl group to the N-terminal amino group of (Sp₂) or K and via its amino group with the C-terminal carboxyl group of (Sp₁) or B₂.

In certain embodiments, the linker may comprise two linking moieties B₁ and B₂.

That is, in certain embodiments, the invention encompasses linkers comprising two bio-orthogonal markergroups and/or non-bio-orthogonal entities. For example, a linker according to the invention may comprise an azide-comprising linking moiety, such as Lys(N₃) or Xaa(N₃), and a sulfhydryl-comprising linking moiety, such as cysteine. In certain embodiments, the linker according to the invention may comprise an azide-comprising linking moiety, such as Lys(N₃) or Xaa(N₃), and a tetrazine-comprising linking moiety, such as a tetrazine-modified amino acid. In certain embodiments, the linker according to the invention may comprise a sulfhydryl-comprising linking moiety, such as cysteine, and a tetrazine-comprising linking moiety, such as a tetrazine-modified amino acid. Linkers comprising two different bio-orthogonal marker groups and/or non-bio-orthogonal entities have the advantage that they can accept two distinct payloads and thus result in antibody-payload conjugates comprising more than one payload.

In such way, an antibody payload ratio of 2+2 may be obtained. Using a second payload may allow for the development of a completely new class of antibody payload conjugates that go beyond current therapeutic approaches with respect to efficacy and potency.

Such embodiments may allow, inter alia, to target two different structures in a cell, like, e.g., the DNA and microtubule. Because some cancers can be resistant to one drug, like e.g., a mirobutule toxin, the DNA-toxin can still kill the cancer cells.

According to another embodiment, two drugs may be used that are only fully potent when they are released at the same time and in the same tissue. This may lead to reduced off-target toxicity in case the antibody is partially degraded in healthy tissues or one drug is pre-maturely lost.

Furthermore, dual-labeled probes may be used for non-invasive imaging and therapy or intra/post-operative imaging/surgery. In such embodiments, a tumor patient may be selected by means of the non-invasive imaging. Then, the tumor may be removed surgically using the other imaging agent (e.g., a fluorescent dye), which helps the surgeon or robot to identify all cancerous tissue during a surgery.

In certain embodiments, one of B₁ and B₂ may be a linking moiety comprising a thiol group, such as cysteine, and the other one of B₁ and B₂ may be a linking moiety comprising an azide moiety, such as Lys(N₃). In such embodiments, two distinct payloads may be coupled to a linker, one via a thiol-maleimide conjugation and the other one via a SPAAC reaction.

In certain embodiments, the linker may comprise two payloads. Linkers comprising only payloads but no linking moieties may be conjugated to an antibody in a one-step process.

It is to be understood that in embodiments where B₁ and B₂ are both payloads, B₁ and B₂ may be identical or may be different in structure. In certain embodiments, linkers comprising one or more payloads may be synthesized chemically. Alternatively, one or more payloads may be coupled to a linking moiety comprised in the linker by any of the methods disclosed herein before the linker is conjugated to an antibody.

In certain embodiments, the linkers of the invention may allow to conjugate two different payloads to the residue Q295 of the C_{H}2 domain of an antibody. Using a second payload allows for the development of a completely new class of antibody-payload conjugates that go beyond current therapeutic approaches with respect to efficacy and potency. Also new application fields are envisioned, for example, dual-type imaging for imaging and therapy or intra-/postoperative surgery (cf. Azhdarinia A. et al., Dual-Labeling Strategies for Nuclear and Fluorescence Molecular Imaging: A Review and Analysis. Mol Imaging Biol. 2012 June; 14(3): 261-276). For example, dual-labeled antibodies encompassing a molecular imaging agent for preoperative positron emission tomography (PET) and a near-infrared fluorescent (NIRF)-dye for guided delineation of surgical margins could greatly enhance the diagnosis, staging, and resection of cancer (cf. Houghton J L. et al., Site-specifically labeled CA19.9-targeted immunoconjugates for the PET, NIRF, and multimodal PET/NIRF imaging of pancreatic cancer. Proc Natl Acad Sci USA. 2015 Dec. 29; 112(52):15850-5). PET and NIRF optical imaging offer complementary clinical applications, enabling the non-invasive whole-body imaging to localize disease and identification of tumor margins during surgery, respectively. However, the generation of such dual-labeled probes up to date has been difficult due to a lack of suitable site-specific methods; attaching two different probes by chemical means results in an almost impossible analysis and reproducibility due to the random conjugation of the probes.

Furthermore, in a study of Levengood M. et al., (Orthogonal Cysteine Protection Enables Homogeneous Multi-Drug Antibody-Drug Conjugates. Angewandte Chemie, Volume 56, Issue 3, Jan. 16, 2017) a dual-drug labeled antibody, having attached two different auristatin toxins (having differing physiochemical properties and exerting complementary anti-cancer activities) imparted activity in cell line and xenograft models that were refractory to ADCs comprised of the individual auristatin components. This suggests that dual-labeled ADCs enable to address cancer heterogeneity and resistance more effectively than the single, conventional ADCs alone. Since one resistance mechanism towards ADCs include the active pumping-out of the cytotoxic moiety from the cancer cell, another dual-drug application may include the additional and simultaneous delivery of a drug that specifically blocks the efflux mechanism of the cytotoxic drug. Such a dual-labeled ADC could thus help to overcome cancer resistance to the ADC more effectively than conventional ADCs.

The term “antibody” herein is used in the broadest sense and specifically covers monoclonal antibodies, polyclonal antibodies, multispecific antibodies (e.g., bispecific antibodies) formed from at least two intact antibodies, and antibody fragments so long as they exhibit the desired biological activity. The terms “antibody” and “antibodies” broadly encompass naturally-occurring forms of antibodies (e.g., IgG, IgA, IgM, IgE).

The antibody is preferably a monoclonal antibody. The antibody can be of human origin, but likewise from mouse, rat, goat, donkey, hamster, or rabbit. In case the conjugate is for therapy, a murine or rabbit antibody may optionally be chimerized or humanized.

Fragments or recombinant variants of antibodies comprising the C_{H}2 domain may be, for example,
antibody formats comprising mere heavy chain domains (shark antibodies/IgNAR (V_{H}-C_{H}1-C_{H}2-C_{H}3-C_{H}4-C_{H}5)₂ or camelid antibodies/hcIgG (V_{H}-C_{H}2-C_{H}3)₂)
scFv-Fc (VH-VL-CH2-CH3)2
Fc fusion peptides, comprising an Fc domain and one or more receptor domains.

The antibody may also be bispecific (e.g., DVD-IgG, crossMab, appended IgG-HC fusion) or biparatopic. See Brinkmann and Kontermann; Bispecific antibodies; Drug Discov Today; 2015; 20(7); p.838-47, for an overview.

In a particular embodiment, the invention relates to the method according to the invention, wherein the antibody is an IgG antibody, in particular an IgG1 antibody.

By “IgG” as used herein is meant a polypeptide belonging to the class of antibodies that are substantially encoded by a recognized immunoglobulin gamma gene. In humans, IgG comprises the subclasses or isotypes IgG1, IgG2, IgG3, and IgG4. In mice, IgG comprises IgG1, IgG2a, IgG2b, IgG3. Full-length IgGs consist of two identical pairs of two immunoglobulin chains, each pair having one light and one heavy chain, each light chain comprising immunoglobulin domains VL and CL, and each heavy chain comprising immunoglobulin domains VH, Cy1 (also called CH1), Cy2 (also called CH2), and Oy3 (also called CH3). In the context of human IgG1, “CH1” refers to positions 118-215, CH2 domain refers to positions 231-340 and CH3 domain refers to positions 341-447 according to the EU index as in Kabat. IgG1 also comprises a hinge domain which refers to positions 216-230 in the case of IgG1.

The antibody used in the method of the invention or the antibody-payload conjugate of the invention may be or comprise any antibody, preferably any IgG type antibody. For example, the antibody may be, without limitation, Brentuximab, Trastuzumab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Sacituzumab, Belantamab, Polatuzumab and Enfortumab.

Thus, in a particular embodiment, the invention relates to the method according to the invention, wherein the antibody is selected from the group consisting of: Brentuximab, Trastuzumab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Sacituzumab, Belantamab, Polatuzumab and Enfortumab.

In a preferred embodiment, the invention relates to the method according to the invention, wherein the antibody is selected from the group consisting of: Brentuximab, Gemtuzumab, Trastuzumab, Inotuzumab, Polatuzumab, Enfortumab, Sacituzumab and Belantamab.

In a more preferred embodiment, the invention relates to the method according to the invention, wherein the antibody is Polatuzumab or Trastuzumab or Enfortumab.

That is, in a particular embodiment, the invention relates to an antibody-linker conjugate, wherein the antibody is Polatuzumab and wherein the linker is any one of the linkers disclosed herein.

In another embodiment, the invention relates to an antibody-linker conjugate, wherein the antibody is Trastuzumab and wherein the linker is any one of the linkers disclosed herein.

In another embodiment, the invention relates to an antibody-linker conjugate, wherein the antibody is Enfortumab and wherein the linker is any one of the linkers disclosed herein.

The antibody for use in the method according to the invention may be a glycosylated antibody, a deglycosylated antibody or an aglycosylated antibody.

That is, in certain embodiments, the antibody may be an IgG antibody that is glycosylated, preferably at residue N297. Thus, in a particular embodiment, the invention relates to the method according to the invention, wherein the IgG antibody is a glycosylated IgG antibody, in particular wherein the IgG antibody is glycosylated at residue N297 (EU numbering) of the C_{H}2 domain.

As discussed herein, IgG antibodies that are glycosylated at residue N297 have several advantages over non-glycosylated antibodies.

However, the antibody may also be a deglycosylated antibody, preferably wherein the glycan at residue N297 has been cleaved off with the enzyme PNGase F. Further, the antibody may be an aglycosylated antibody, preferably wherein residue N297 has been replaced with a non-asparagine residue. Methods for deglycosylating antibodies and for generating aglycosylated antibodies are known in the art.

In certain embodiments, the linker of the invention may be conjugated to an endogenous Gln residue in the Fc domain of an antibody or to a Gln residue that has been introduced into the antibody by means of molecular engineering.

Thus, in a particular embodiment, the invention relates to the method according to the invention, wherein the Gln residue to which the linker is conjugated is comprised in an Fc domain of the antibody, in particular wherein the Gln residue to which the linker is conjugated is Gln residue Q295 (EU numbering) of the C_{H}2 domain of an IgG antibody.

The linkers of the invention may be conjugated to any Gln residue in the Fc domain of an antibody that can serve as a substrate for a transglutaminase. Typically, the term Fc domain as used herein refers to the last two constant region immunoglobulin domains of IgA, IgD and IgG (C_{H}2 and C_{H}3) and the last three constant region domains of IgE, IgY and IgM (C_{H}2, C_{H}3 and C_{H}4). That is, the linker according to the invention may be conjugated to the C_{H}2, C_{H}3 and, where applicable, C_{H}4 domains of the antibody.

In certain embodiments, the endogenous Gln residue may be Gln residue Q295 (EU numbering) of the CH2 domain of an IgG antibody. Thus, in a particular embodiment, the invention relates to the method according to the invention, wherein the Gln residue in the Fc domain of the antibody is Gln residue Q295 (EU numbering) of the CH2 domain of an IgG antibody. In a particularly preferred embodiment, the Gln residue in the Fc domain of the antibody is Gln residue Q295 (EU numbering) of the CH2 domain of a glycosylated IgG antibody, in particular a glycosylated IgG antibody having an unmodified constant region.

It is important to understand that Q295 is an extremely conserved amino acid residue in IgG type antibodies. It is conserved in human IgG1, 2, 3, 4, as well as in rabbit and rat antibodies amongst others. Hence, being able to use Q295 is a considerable advantage for making therapeutic antibody-payload conjugates, or diagnostic conjugates where the antibody is often of non-human origin. The method according to the invention does hence provide an extremely versatile and broadly applicable tool. Even though residue Q295 is extremely conserved among IgG type antibodies, some IgG type antibodies do not possess this residue, such as mouse and rat IgG2a antibodies. Thus, it is to be understood that the antibody used in the method of the present invention is preferably an IgG type antibody comprising residue Q295 (EU numbering) of the C_{H}2 domain.

Further, it has been shown that engineered conjugates using Q295 for payload attachment demonstrate good pharmacokinetics and efficacy (Lhospice et al., Site-Specific Conjugation of Monomethyl Auristatin E to Anti-Cd30 Antibodies Improves Their Pharmacokinetics and Therapeutic Index in Rodent Models, Mol Pharm; 2015; 12(6), p.1863-1871), and are capable of carrying even unstable toxins prone for degradation (Dorywalska et al.; Site-Dependent Degradation of a Non-Cleavable Auristatin-Based Linker-Payload in Rodent Plasma and Its Effect on ADC Efficacy. PLoS ONE; 2015; 10(7): e0132282). It is thus expected that similar effects will be seen with this site-specific method since the same residue is modified, but of glycosylated antibodies. Glycosylation may further contribute to overall ADC stability, removal of the glycan moieties as with the mentioned approaches has been shown to result in less-stable antibodies (Zheng et al.; The impact of glycosylation on monoclonal antibody conformation and stability. Mabs-Austin; 2011, 3(6), p.568-576).

In the literature discussing the conjugation of linkers to a C_{H}2 Gln residue by means of a transglutaminase, the focus has been on small, low-molecular weight substrates. However, in the prior art literature, to accomplish such conjugation, a deglycosylation step in position N297, or the use of an aglycosylated antibody, is always described as necessary (WO 2015/015448; WO 2017/025179; WO 2013/092998).

Quite surprisingly, and against all expectations, however, site-specific conjugation to Q295 of glycosylated antibodies is indeed efficiently possible by using the above discussed linker structure. In particular, coupling of linkers comprising toxin molecules was achieved with a conjugation efficiency greater than 80%.

Even though Q295 is very close to N297, which is, in its native state, glycosylated, the method according to the invention, using the specified linker, still allows the conjugation of the linker or payload thereto.

As shown, the method according to the invention does not require an upfront enzymatic deglycosylation of N297, nor the use of an aglycosylated antibody, nor a substitution of N297 against another amino acid, nor the introduction of a T299A mutation to prevent glycosylation.

These two points provide significant advantages under manufacturing aspects. An enzymatic deglycosylation step is undesired under GMP aspects, because it has to be made sure that the both the deglycosylation enzyme (e.g., PNGase F) as well as the cleaved glycan have to be removed from the medium.

Furthermore, no genetic engineering of the antibody for payload attachment is necessary, so that sequence insertions which may increase immunogenicity and decrease the overall stability of the antibody can be avoided.

The substitution of N297 against another amino acid has unwanted effects, too, because it may affect the overall stability of the entire Fc domain (Subedi et al, The Structural Role of Antibody N-Glycosylation in Receptor Interactions. Structure 2015, 23 (9), 1573-1583), and the efficacy of the entire conjugate as a consequence that can lead to increased antibody aggregation and a decreased solubility (Zheng et al.; The impact of glycosylation on monoclonal antibody conformation and stability. Mabs-Austin 2011, 3 (6), 568-576) that particularly gets important for hydrophobic payloads such as PBDs. Further, the glycan that is present at N297 has important immunomodulatory effects, as it triggers antibody dependent cellular cytotoxicity (ADCC) and the like. These immunomodulatory effects would get lost upon deglycosylation or any of the other approaches discussed above to obtain an aglycosylated antibody. Further, any sequence modification of an established antibody can also lead to regulatory problems, which is problematic because often times an accepted and clinically validated antibody is used as a starting point for ADC conjugation.

Hence, the method according to the invention allows to easily and without disadvantages make stoichiometrically well-defined ADCs with site specific payload binding.

In view of the above, it is stated that the method of the present invention is preferably used for the conjugation of an IgG antibody at residue Q295 (EU numbering) of the C_{H}2 domain of the antibody, wherein the antibody is glycosylated at residue N297 (EU numbering) of the C_{H}2 domain. However, it is expressly stated that the method of the invention also encompasses the conjugation of deglycosylated or aglycosylated antibodies at residue Q295 or any other suitable Gln residue of the antibody, wherein the Gln residue may be an endogenous Gln residue or a Gln residue that has been introduced by molecular engineering.

Thus, in a particular embodiment, the invention relates to the method according to the invention, wherein the Gln residue to which the linker is conjugated has been introduced into the heavy or light chain of the antibody by molecular engineering.

The term “molecular engineering,” as used herein, refers to the use of molecular biology methods to manipulate nucleic acid sequences. Within the present invention, molecular engineering may be used to introduce Gln residues into the heavy or light chain of an antibody.

In general, two different strategies to introduce Gln residues into the heavy or light chain of an antibody are envisioned within the present invention. First, single residues of the heavy or light chain of an antibody may be substituted with a Gln residue. Second, Gln-containing peptide tags consisting of two or more amino acid residues may be integrated into the heavy or light chain of an antibody. For that, the peptide tag may either be integrated into an internal position of the heavy or light chain, that is, between two existing amino acid residues of the heavy or light chain or by replacing them, or the peptide tag may be fused (appended) to the N- or C-terminal end of the heavy or light chain of the antibody.

For example, an amino residue of the heavy or light chain of an antibody may be substituted with a Gln residue, provided that the resulting antibody can be conjugated with the linkers of the invention by a transglutaminase. In certain embodiments, the antibody is an antibody wherein amino acid residue N297 (EU numbering) of the C_{H}2 domain of an IgG antibody is substituted, in particular wherein the substitution is an N297Q substitution. Antibodies comprising an N297Q mutation may be conjugated to more than one linker per heavy chain of the antibody. For example, antibodies comprising an N297Q mutation may be conjugated to four linkers, wherein one linker is conjugated to residue Q295 of the first heavy chain of the antibody, one linker is conjugated to residue N297Q of the first heavy chain of the antibody, one linker is conjugated to residue Q295 of the second heavy chain of the antibody and one linker is conjugated to residue N297Q of the second heavy chain of the antibody. The skilled person is aware that replacement of residue N297 of an IgG antibody with a Gln residue results in an aglycosylated antibody.

Thus, in a particular embodiment, the invention relates to the method according to the invention, wherein the Gln residue that has been introduced into the heavy or light chain of the antibody by molecular engineering is N297Q (EU numbering) of the C_{H}2 domain of an aglycosylated IgG antibody.

In a particular embodiment, the invention relates to the method according to the invention, wherein the Gln residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.

Instead of substituting single amino acid residues of an antibody, peptide tags comprising a Gln residue that is accessible for a transglutaminase may be introduced into the heavy or light chain of the antibody. Such peptide tags may be fused to the N- or C-terminus of the heavy or light chain of the antibody. Alternatively, peptide tags may be inserted into the heavy or light chain of an antibody at a suitable position. Preferably, peptide tags comprising a transglutaminase-accessible Gln residue are fused to the C-terminus of the heavy chain of the antibody. Even more preferably, the peptide tags comprising a transglutaminase-accessible Gln residue are fused to the C-terminus of the heavy chain of an IgG antibody. Several peptide tags that may be fused to the C-terminus of the heavy chain of an antibody and serve as substrate for a transglutaminase are described in WO 2012/059882 and WO 2016/144608.

Thus, in a particular embodiment, the invention relates to the method according to the invention, wherein the peptide comprising the Gln residue has been fused to the C-terminal end of the heavy chain of the antibody.

Exemplary peptide tags that may be introduced into the heavy or light chain of an antibody, in particular fused to the C-terminus of the heavy chain of the antibody, are LLQGG (SEQ ID NO:1), LLQG (SEQ ID NO:2), LSLSQG (SEQ ID NO:3), GGGLLQGG (SEQ ID NO:4), GLLQG (SEQ ID NO:5), LLQ, GSPLAQSHGG (SEQ ID NO:6), GLLQGGG (SEQ ID NO:7), GLLQGG (SEQ ID NO:8), GLLQ (SEQ ID NO:9), LLQLLQGA (SEQ ID NO:10), LLQGA (SEQ ID NO:11), LLQYQGA (SEQ ID NO:12), LLQGSG (SEQ ID NO:13), LLQYQG (SEQ ID NO:14), LLQLLQG (SEQ ID NO:15), SLLQG (SEQ ID NO:16), LLQLQ (SEQ ID NO:17), LLQLLQ (SEQ ID NO:18), LLQGR (SEQ ID NO:19), EEQYASTY (SEQ ID NO:20), EEQYQSTY (SEQ ID NO:21), EEQYNSTY (SEQ ID NO:22), EEQYQS (SEQ ID NO:23), EEQYQST (SEQ ID NO:24), EQYQSTY (SEQ ID NO:25), QYQS (SEQ ID NO:26), QYQSTY (SEQ ID NO:27), YRYRQ (SEQ ID NO:28), DYALQ (SEQ ID NO:29), FGLQRPY (SEQ ID NO:30), EQKLISEEDL (SEQ ID NO:31), LQR and YQR.

The skilled person is aware of methods to substitute amino acid residues of antibodies or to introduce peptide tags into antibodies, for example by methods of molecular cloning as described in Sambrook, Joseph. (2001). Molecular cloning: a laboratory manual. Cold Spring Harbor, N.Y.: Cold Spring Harbor Laboratory Press.

In general, the skilled person is aware of methods to determine at which position of an antibody a linker is conjugated. For example, the conjugation site may be determined by proteolytic digestion of the antibody-payload conjugate and LC-MS analysis of the resulting fragments. For example, samples may be deglycosylated with GlyciNATOR (Genovis) according to the instruction manual and subsequently digested with trypsin gold (mass spectrometry grade, Promega), respectively. Therefore, 1 μg of protein may be incubated with 50 ng trypsin at 37° C. overnight. LC-MS analysis may be performed using a nanoAcquity HPLC system coupled to a Synapt-G2 mass spectrometer (Waters). For that, 100 ng peptide solution may be loaded onto an Acquity UPLC Symmetry C18 trap column (Waters, part no. 186006527) and trapped with 5 μL/min flow rate at 1% buffer A (Water, 0.1% formic acid) and 99% buffer B (acetonitrile, 0.1% formic acid) for 3 min. Peptides may then be eluted with a linear gradient from 3% to 65% Buffer B within 25 min. Data may be acquired in resolution mode with positive polarity and in a mass range from 50 to 2000 m/z. Other instrument settings may be as follows: capillary voltage 3.2 kV, sampling cone 40 V, extraction cone 4.0 V, source temperature 130° C., cone gas 35 L/h, nano flow gas 0.1 bar, and purge gas 150 L/h. The mass spectrometer may be calibrated with [Glu1]-Fibrinopeptide.

Further, the skilled person is aware of methods to determine the drug-to-antibody (DAR) ratio or payload-to-antibody ratio of an antibody-payload construct. For example, the DAR may be determined by hydrophobic interaction chromatography (HIC) or LC-MS.

For hydrophobic interaction chromatography (HIC), samples may be adjusted to 0.5 M ammonium sulfate and assessed via a MAB PAK HIC Butyl column (5 μm, 4.6×100 mm, Thermo Scientific) using a full gradient from A (1.5 M ammonium sulfate, 25 mM Tris HCl, pH 7.5) to B (20% isopropanol, 25 mM Tris HCl, pH 7.5) over 20 min at 1 mL/min and 30° C. Typically, 40 μg sample may be used and signals may be recorded at 280 nm. Relative HIC retention times (HIC-RRT) may be calculated by dividing the absolute retention time of the ADC DAR 2 species by the retention time of the respective unconjugated mAb.

For LC-MS DAR determination, ADCs may be diluted with NH₄HCO₃ to a final concentration of 0.025 mg/mL. Subsequently, 40 μL of this solution may be reduced with 1 μL TCEP (500 mM) for 5 min at room temperature and then alkylated by adding 10 μL chloroacetamide (200 mM), followed by overnight incubation at 37° C. in the dark. For reversed phase chromatography, a Dionex U3000 system in combination with the software Chromeleon may be used. The system may be equipped with a RP-1000 column (1000 Å, 5 μm, 1.0×100 mm, Sepax) heated to 70° C., and an UV-detector set to a wavelength of 214 nm. Solvent A may consist of water with 0.1% formic acid and solvent B may comprise 85% acetonitrile with 0.1% formic acid. The reduced and alkylated sample may be loaded onto the column and separated by a gradient from 30-55% solvent B over the course of 14 min. The liquid chromatography system may be coupled to a Synapt-G2 mass spectrometer for identification of the DAR species. The capillary voltage of the mass spectrometer may be set to 3 kV, the sampling cone to 30 V and the extraction cone may add up to a value of 5 V. The source temperature may be set to 150° C., the desolvation temperature to 500° C., the cone gas to 20 l/h, the desolvation gas to 600 l/h, and the acquisition may be made in positive mode in a mass range from 600-5000 Da with 1 s scan time. The instrument may be calibrated with sodium iodide. Deconvolution of the spectra may be performed with the MaxEnt1 algorithm of MassLynx until convergence. After assignment of the DAR species to the chromatographic peaks, the DAR may be calculated based on the integrated peak areas of the reversed phase chromatogram.

In a particular embodiment, the invention relates to the method according to the invention, wherein the linker is conjugated to the γ-carboxamide group of the Gln residue comprised in the antibody.

That is, the linker according to the invention is preferably conjugated to the amide group in the side chain of a Gln residue comprised in the antibody, preferably any one of the Gln residues disclosed herein, more preferably Gln residue Q295 (EU numbering).

In a particular embodiment, the invention relates to the method according to the invention, wherein the linker is suitable for conjugation to a glycosylated antibody with a conjugation efficiency of at least 20%, 30%, 40%, 50%, 60%, 70%, 75%, 80%, 85%, 90% or 95%.

That is, in certain embodiments, the linker may be a linker that can be conjugated to a glycosylated antibody with an efficiency of at least 20%, 30%, 40%, 50%, 60%, 70%, 75%, 80%, 85%, 90% or 95%. In a preferred embodiment, the linker may be a linker that can be conjugated to a glycosylated antibody with an efficiency of at least 70%. In another preferred embodiment, the linker may be a linker that can be conjugated to a glycosylated antibody with an efficiency of at least 75%. In another preferred embodiment, the linker may be a linker that can be conjugated to a glycosylated antibody with an efficiency of at least 80%. In another preferred embodiment, the linker may be a linker that can be conjugated to a glycosylated antibody with an efficiency of at least 85%. In another preferred embodiment, the linker may be a linker that can be conjugated to a glycosylated antibody with an efficiency of at least 90%. In another preferred embodiment, the linker may be a linker that can be conjugated to a glycosylated antibody with an efficiency of at least 95%. Preferably, the glycosylated antibody is a glycosylated IgG antibody, more preferably an IgG antibody that is glycosylated at residue N297 (EU numbering).

The skilled person is aware of methods to determine the conjugation efficiency of an antibody with a specific linker. For example, the conjugation efficiency may be determined as described herein. That is, an antibody, in particular an IgG1 antibody, may be incubated under conditions as defined herein. After the incubation period, the conjugation efficiency may be determined by LC-MS analysis under reducing conditions. The transglutaminase may be a microbial transglutaminase (MTG) from Streptomyces mobaraensis that is available from Zedira (Germany). A suitable buffer may be a Tris, MOPS, HEPES, PBS or BisTris buffer. However, it is to be understood that the choice of the buffer system may vary and depend to a large extent on the chemical properties of the linker. However, the skilled person is capable of identifying the optimal buffer conditions based on the disclosure of the present invention. Alternatively, the conjugation efficiency may be determined as described in Spycher et al. (Dual, Site-Specific Modification of Antibodies by Using Solid-Phase Immobilized Microbial Transglutaminase, ChemBioChem 2019 18(19):1923-1927) and analyzed as in Benjamin et al. (Thiolation of Q295: Site-Specific Conjugation of Hydrophobic Payloads without the Need for Genetic Engineering, Mol. Pharmaceutics 2019, 16: 2795-2807).

In certain embodiments, antibodies may be conjugated as described in Example 1. That is, 5 mg/ml of native, glycosylated monoclonal antibody may be incubated for 24 hours at 37° C. in 50 mM Tris pH 7.6 comprising microbial transglutaminase (MTG, Zedira) at a concentration of 5 U/mg antibody and 5 molar equivalents of the indicated linker-payload in a rotating thermomixer.

In a particular embodiment, the invention relates to the method according to the invention, wherein the microbial transglutaminase is derived from a Streptomyces species, in particular Streptomyces mobaraensis.

That is, the microbial transglutaminase used in the method of the invention may be derived from a Streptomyces species, in particular from Streptomyces mobaraensis, preferentially with a sequence identity of 80% to the native enzyme. Accordingly, the MTG may be a native enzyme or may be an engineered variant of a native enzyme.

One such microbial transglutaminase is commercially available from Zedira (Germany). It is recombinantly produced in E. coli. Streptomyces mobaraensis transglutaminase has an amino acid sequence as disclosed in SEQ ID NO:32. S. mobaraensis MTG variants with other amino acid sequences have been reported and are also encompassed by this invention (SEQ ID NO:33 and 34).

In another embodiment, a microbial transglutaminase from Streptomyces ladakanum (formerly known as Streptoverticillium ladakanum) may be used. Streptomyces ladakanum transglutaminase (U.S. Pat. No. 6,660,510 B2) has an amino acid sequence as disclosed in SEQ ID NO:35.

Both of the above transglutaminases may be sequence modified. In several embodiments, transglutaminases may be used which have 80%, 85%, 90% or 95% or more sequence identity with any one of SEQ ID NO:32-35.

Another suitable microbial transglutaminase is commercially from Ajinomoto, called ACTIVA TG. In comparison to the transglutaminase from Zedira, ACTIVA TG lacks 4 N-terminal amino acids, but has similar activity.

Further microbial transglutaminases which may be used in the context of the present invention are disclosed in Kieliszek and Misiewicz (Folia Microbiol (Praha). 2014; 59(3): 241-250), WO 2015/191883 Å1, WO 2008/102007 Å1 and US 2010/0143970, the content of which is fully incorporated herein by reference.

In certain embodiments, a mutant variant of a microbial transglutaminase may be used for the conjugation of a linker to an antibody. That is, the microbial transglutaminase that is used in the method of the present invention may be a variant of S. mobaraensis transgluatminase as set forth in SEQ ID NOs: 32 or 33. In certain embodiments, the recombinant S. morabaensis transglutaminase as set forth in SEQ ID NO:32 may comprise the mutation G254D. In certain embodiments, the recombinant S. morabaensis transglutaminase as set forth in SEQ ID NO:32 may comprise the mutations G254D and E304D. In certain embodiments, the recombinant S. morabaensis transglutaminase as set forth in SEQ ID NO:32 may comprise the mutations D8E and G254D. In certain embodiments, the recombinant S. morabaensis transglutaminase as set forth in SEQ ID NO:32 may comprise the mutations E124A and G254D. In certain embodiments, the recombinant S. morabaensis transglutaminase as set forth in SEQ ID NO:32 may comprise the mutations A216D and G254D. In certain embodiments, the recombinant S. morabaensis transglutaminase as set forth in SEQ ID NO:32 may comprise the mutations G254D and K331T.

In a particular embodiment, the invention relates to an antibody-linker conjugate which has been produced with a method according to the invention.

That is, the invention relates to an antibody-linker conjugate which has been generated with any of the aforementioned steps.

Further, the invention relates to pharmaceutical compositions comprising the antibody-linker conjugate according to the invention.

Thus, in a particular embodiment, the invention relates to a pharmaceutical composition comprising the antibody-linker conjugate according to the invention, in particular wherein the antibody-linker conjugate comprises at least one payload, and at least one pharmaceutically acceptable ingredient.

It is to be understood that the pharmaceutical composition may comprises an antibody-payload conjugate that has been produced with the one-step or two-step process disclosed herein.

The type of payload that is comprised in the antibody-payload construct comprised in the pharmaceutical composition depends on the use of the pharmaceutical composition. In embodiments where the pharmaceutical composition is used for the treatment of a disease, the payload is preferably a drug. If the disease is a neoplastic disease, the payload is preferably a toxin. In embodiments where the pharmaceutical composition is used in diagnostics, the payload is preferably an imaging agent.

In a particular embodiment, the invention relates to a pharmaceutical composition according to the invention comprising at least one additional therapeutically active agent.

The pharmaceutical composition according to the invention may comprise at least one pharmaceutically acceptable ingredient.

A pharmaceutically acceptable ingredient refers to an ingredient in a pharmaceutical formulation, other than an active ingredient, which is nontoxic to a subject. A pharmaceutically acceptable ingredient includes, but is not limited to, a buffer, excipient, stabilizer, or preservative.

Pharmaceutical formulations of the antibody-linker conjugates described herein are prepared by mixing such conjugates having the desired degree of purity with one or more optional pharmaceutically acceptable ingredients (Flemington's Pharmaceutical Sciences 16th edition, Oslo, A. Ed. (1980)), in the form of lyophilized formulations or aqueous solutions. Pharmaceutically acceptable ingredients are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn protein complexes); and/or non-ionic surfactants such as polyethylene glycol (PEG). Exemplary pharmaceutically acceptable ingredients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. For example, a sHASEGP may be combined with one or more additional glycosaminoglycanases such as chondroitinases.

In a particular embodiment, the invention relates to the antibody-linker conjugate according to the invention, in particular wherein the antibody-linker conjugate comprises at least one payload, or the pharmaceutical composition according to the invention for use in therapy and/or diagnostics.

That is, the antibody-linker conjugate or the pharmaceutical composition according to the invention may be used in the treatment of a subject or in diagnosing a disease or condition in a subject. An individual or subject is preferably a mammal. Mammals include, but are not limited to, domesticated animals (e.g., cows, sheep, cats, dogs, and horses), primates (e.g., humans and non-human primates such as macaques), rabbits, and rodents (e.g., mice and rats). In certain embodiments, the individual or subject is a human. When an antibody-linker conjugate or a pharmaceutical composition comprising an antibody-linker conjugate according to the invention is used in therapy, it is preferred that the linker comprises a drug. When an antibody-linker conjugate or a pharmaceutical composition comprising an antibody-linker conjugate according to the invention is used in diagnostics, it is preferred that the linker comprises at least one imaging agent.

In a particular embodiment, the invention relates to the antibody-linker conjugate according to the invention, in particular wherein the antibody-linker conjugate comprises at least one payload, or the pharmaceutical composition according to the invention for use in the treatment of a patient
suffering from,
being at risk of developing, and/or
being diagnosed for
   a neoplastic disease, a neurological disease, an autoimmune disease, an inflammatory disease or an infectious disease.

In a particular embodiment, the invention relates to the antibody-linker conjugate according to the invention, in particular wherein the antibody-linker conjugate comprises at least one payload, or the pharmaceutical composition according to the invention for use in treatment of a patient suffering from a neoplastic disease.

The term “neoplastic disease” as used herein refers to a condition characterized by uncontrolled, abnormal growth of cells. Neoplastic diseases include cancer. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, kidney cancer, vulval cancer, thyroid cancer, hepatic carcinoma, skin cancer, melanoma, brain cancer, ovarian cancer, neuroblastoma, myeloma, various types of head and neck cancer, acute lymphoblastic leukemia, acute myeloid leukemia, Ewing sarcoma and peripheral neuroepithelioma. Preferred cancers include liver cancer, lymphoma, acute lymphoblastic leukemia, acute myeloid leukemia, Ewing sarcoma and peripheral neuroepithelioma.

That is, the antibody-linker conjugates according to the invention are preferably used for the treatment of cancer. As such, in certain embodiments, the antibody-linker conjugates according to invention comprise an antibody that specifically binds to an antigen that is present on a tumor cell. In certain embodiments, the antigen may be an antigen on the surface of a tumor cell. In certain embodiments, the antigen on the surface of the tumor cell may be internalized into the cell together with the antibody-linker conjugate upon binding of the antibody-linker conjugate to the antigen.

If the antibody-linker conjugate according to the invention is used in the treatment of cancer, it is preferred that the antibody-linker conjugate comprises at least one payload that has the potential to kill or inhibit the proliferation of the tumor cell to which the antibody-linker conjugate binds to. In certain embodiments, the at least one payload exhibits its cytotoxic activity after the antibody-linker conjugate has been internalized into the tumor cell. In certain embodiments, the at least one payload is a toxin.

The inflammatory disease may be an autoimmune disease. The infectious disease may be a bacterial infection or a viral infection.

In certain embodiments, the antibody-linker conjugate and/or the pharmaceutical composition according to the invention may be used in the treatment of B-cell-associated cancer.

Thus, in a particular embodiment, the invention relates to the antibody-linker conjugate or the pharmaceutical composition for use according to the invention, wherein the antibody-linker conjugate comprised in the pharmaceutical composition comprises Polatuzumab and wherein the neoplastic disease is a B-cell associated cancer.

For this, it is preferred that the antibody-linker conjugate comprises an anti-CD79b antibody as disclosed herein, preferably wherein the anti-CD79b antibody is internalized into a target cell upon binding to CD79b. In certain embodiments, the anti-CD79b antibody is Polatuzumab with a heavy chain as set forth in SEQ ID NO:36 and a light chain as set forth in SEQ ID NO:37. Further, it is preferred that the antibody-linker conjugate comprises at least one toxin.

A B-cell associated cancer may be any one selected from a group consisting of: high, intermediate and low grade lymphomas (including B cell lymphoma such as, for example, mucosa-associated lymphoid tissue B cell lymphoma and non-Hodgkin's lymphoma (NHL), mantle cell lymphoma, Burkitt's lymphoma, small lymphocytic lymphoma, marginal Zone lymphoma, diffuse large B cell lymphoma, follicular lymphoma, and Hodgkin's lymphoma and T cell lymphomas) and leukemias (including secondary leukemia, chronic lymphocytic leukemia (CLL), such as B cell leukemia (CD5+B lymphocytes), myeloid leukemia, such as acute myeloid leukemia, chronic myeloid leukemia, lymphoid leukemia, such as acute lymphoblastic leukemia (ALL) and myelodysplasia), and other hematological and/or B cell—or T-cell-associated cancers, including cancers of additional hematopoietic cells, including polymorphonuclear leukocytes, such as basophils, eosinophils, neutrophils and monocytes, dendritic cells, platelets, erythrocytes and natural killer cells. Also included are cancerous B cell proliferative disorders selected from the following: lymphoma, non-Hodgkins lymphoma (NHL) aggressive NHL, relapsed aggressive NHL, relapsed indolent NHL, refractory NHL, refractory indolent NHL, chronic lymphocytic leukemia (CLL), Small lymphocytic lymphoma, leukemia, hairy cell leukemia (HCL), acute lymphocytic leukemia (ALL), and mantle cell lymphoma.

In a particular embodiment, the invention relates to the antibody-linker conjugate, or the pharmaceutical composition for use according to the invention, wherein the B-cell associated cancer is non-Hodgkin lymphoma, in particular wherein the B-cell associated cancer is diffuse large B-cell lymphoma.

Further, the anti-CD79b antibody-linker conjugate and/or the pharmaceutical composition comprising an anti-CD79b antibody-linker conjugate may be used in conjunction with other therapies that are suitable for the treatment of B-cell-associated cancer.

Thus, in a particular embodiment, the invention relates to the antibody-linker conjugate or the pharmaceutical composition for use according to the invention, wherein the antibody-linker conjugate or the pharmaceutical composition is administered in combination with bendamustine and/or rituximab.

It is to be understood that the antibody-linker conjugate or the pharmaceutical composition does not necessarily have to be administered at the same time as the additional therapeutic agent, such as bendamustine and/or rituximab. Instead the antibody-linker conjugate or the pharmaceutical composition may be administered with a different administration schedule and, consequently, on different days as other therapeutic agents that are used for the treatment of the same disease.

In certain embodiments, the antibody-linker conjugate and/or the pharmaceutical composition according to the invention may be used in the treatment of HER2-positive cancers.

Thus, in a particular embodiment, the invention relates to the antibody-linker conjugate or the pharmaceutical composition for use according to the invention, wherein the antibody-linker conjugate comprised in the pharmaceutical composition comprises Trastuzumab and wherein the neoplastic disease is a HER2-positive cancer, in particular HER2-positive breast, gastric, ovarian or lung cancer.

For this, it is preferred that the antibody-linker conjugate comprises an anti-HER2/neu antibody as disclosed herein, preferably wherein the anti-HER2/neu antibody is internalized into a target cell upon binding to HER2/neu. In certain embodiments, the anti-HER2/neu antibody is Trastuzumab with a heavy chain as set forth in SEQ ID NO:38 and a light chain as set forth in SEQ ID NO:39. Further, it is preferred that the antibody-linker conjugate comprises at least one toxin.

A HER2-positive cancer, as used herein, may be, without limitation HER2-positive breast, gastric, ovarian or lung cancer. The skilled person is able do determine whether a cancer is a HER2-positive cancer. For example, tumor cells may be isolated in a biopsy and the presence of HER2/neu may be determined with any method known in the art.

Further, the anti-HER2/neu antibody-linker conjugate and/or the pharmaceutical composition comprising an anti-HER2/neu antibody-linker conjugate may be used in conjunction with other therapies that are suitable for the treatment of HER2-positive cancers.

Thus, in a particular embodiment, the invention relates to the antibody-linker conjugate or the pharmaceutical composition for use according to the invention, wherein the antibody-linker conjugate or the pharmaceutical composition is administered in combination with lapatinib, capecitabine and/or a taxane.

It is to be understood that the antibody-linker conjugate or the pharmaceutical composition does not necessarily have to be administered at the same time as the additional therapeutic agent, such as lapatinib, capecitabine and/or a taxane. Instead the antibody-linker conjugate or the pharmaceutical composition may be administered with a different administration schedule and, consequently, on different days as other therapeutic agents that are used for the treatment of the same disease.

In certain embodiments, the antibody-linker conjugate and/or the pharmaceutical composition according to the invention may be used in the treatment of Nectin-4-positive cancers.

Thus, in a particular embodiment, the invention relates to the antibody-linker conjugate or the pharmaceutical composition for use according to the invention, wherein the antibody-linker conjugate comprised in the pharmaceutical composition comprises Enfortumab or an Enfortumab variant and wherein the neoplastic disease is a Nectin-4 positive cancer, in particular Nectin-4 positive pancreatic cancer, lung cancer, bladder cancer or breast cancer.

For this, it is preferred that the antibody-linker conjugate comprises an anti-Nectin-4 antibody as disclosed herein, preferably wherein the anti-Nectin-4 antibody is internalized into a target cell upon binding to Nectin-4. In certain embodiments, the anti-Nectin-4 antibody is Enfortumab with a heavy chain as set forth in SEQ ID NO:40 or SEQ ID NO:42 and a light chain as set forth in SEQ ID NO:41. Further, it is preferred that the antibody-linker conjugate comprises at least one toxin.

A Nectin-4-positive cancer, as used herein, may be, without limitation Nectin-4-positive pancreatic cancer, lung cancer, bladder cancer or breast cancer. The skilled person is able do determine whether a cancer is a Nectin-4-positive cancer. For example, tumor cells may be isolated in a biopsy and the presence of Nectin-4 may be determined with any method known in the art.

Further, the anti-Nectin-4 antibody-linker conjugate and/or the pharmaceutical composition comprising an anti-Nectin-4 antibody-linker conjugate may be used in conjunction with other therapies that are suitable for the treatment of Nectin-4-positive cancers.

Thus, in a particular embodiment, the invention relates to the antibody-linker conjugate or the pharmaceutical composition for use according to the invention, wherein the antibody-linker conjugate or the pharmaceutical composition is administered in combination with a cisplatin-based chemotherapeutic agent and/or Pembrolizumab.

It is to be understood that the antibody-linker conjugate or the pharmaceutical composition does not necessarily have to be administered at the same time as the additional therapeutic agent, such as the cisplatin-based chemotherapeutic agent and/or Pembrolizumab. Instead the antibody-linker conjugate or the pharmaceutical composition may be administered with a different schedule and, consequently, on different days as other therapeutic agents that are used for the treatment of the same disease.

In a particular embodiment, the invention relates to the use of the antibody-linker conjugate according to the invention, in particular wherein the antibody-linker conjugate comprises at least one payload, or the pharmaceutical composition according to the invention for the manufacture of a medicament for the treatment of a patient
suffering from,
being at risk of developing, and/or
being diagnosed for
   a neoplastic disease, neurological disease, an autoimmune disease, an inflammatory disease or an infectious disease.

In a particular embodiment, the invention relates to a method of treating or preventing a neoplastic disease, said method comprising administering to a patient in need thereof the antibody-linker conjugate according to the invention, in particular wherein the antibody-linker conjugate comprises at least one payload, or the pharmaceutical composition according to the invention.

In a particular embodiment, the invention relates to the antibody-linker conjugate according to the invention, in particular wherein the antibody-linker conjugate comprises at least one payload, or the pharmaceutical composition according to the invention for use in pre-, intra- or post-operative imaging.

That is, the antibody-linker conjugate according to the invention may be used in medical imaging. For that, the antibody-linker conjugate may be visualized while binding to a specific target molecule, cell or tissue. Different techniques are known in the art to visualize particular payloads. For example, if the payload is a radionuclide, the molecules, cells, or tissues to which the antibody-linker conjugate binds may be visualized by PET or SPECT. If the payload is a fluorescent dye, the molecules, cells, or tissues to which the antibody-linker conjugate binds may be visualized by fluorescence imaging. In certain embodiments, the antibody-linker conjugate according to the invention comprises two different payloads, for example a radionuclide and a fluorescent dye. In this case, the molecule, cell or tissue to which the antibody-linker conjugate binds may be visualized using two different and/or complementary imaging techniques, for example PET/SPECT and fluorescence imaging.

The antibody-linker conjugate may be used for pre- intra- and/or post-operative imaging.

Pre-operative imaging encompasses all imaging techniques that may be performed before a surgery to make specific target molecules, cells or tissues visible when diagnosing a certain disease or condition and, optionally, to provide guidance for a surgery. Preoperative imaging may comprise a step of making a tumor visible by PET or SPECT before a surgery is performed by using an antibody-linker conjugate that comprises an antibody that specifically binds to an antigen on the tumor and is conjugated to a payload that comprises a radionuclide.

Intra-operative imaging encompasses all imaging techniques that may be performed during a surgery to make specific target molecules, cells or tissues visible and thus provide guidance for the surgery. In certain embodiments, an antibody-linker conjugate comprising a near-infrared fluorescent dye may be used to visualize a tumor during surgery by near-infrared fluorescent imaging. Intraoperative imaging allows the surgeon to identify specific tissues, for example tumor tissue, during surgery and thus may allow complete removal of tumor tissue.

Post-operative imaging encompasses all imaging techniques that may be performed after a surgery to make specific target molecules, cells or tissues visible and to evaluate the result of the surgery. Post-operative imaging may be performed similarly as pre-operative surgery.

In certain embodiments, the invention relates to antibody-linker conjugates comprising two or more different payloads. For example, the antibody-linker conjugate may comprise a radionuclide and a near-infrared fluorescent dye. Such an antibody-payload conjugate may be used for imaging by PET/SPECT and near-infrared fluorescent imaging. The advantage of such an antibody is that it may be used to visualize the target tissue, for example a tumor before and after a surgery by PET or SPECT. At the same time, the tumor may be visualized during the surgery by near-fluorescent infrared imaging.

In a particular embodiment, the invention relates to the antibody-linker conjugate according to the invention, in particular wherein the antibody-linker conjugate comprises at least one payload, or the pharmaceutical composition according to the invention for use in intraoperative imaging-guided cancer surgery.

As mentioned above, the antibody-linker conjugate of the invention may be used to visualize a target molecule, cell or tissue and to guide a surgeon or robot during a surgery. That is, the antibody-linker conjugate may be used to visualize tumor tissue during a surgery, for example by near-infrared imaging and to allow complete removal of the tumor tissue.

The antibody-linker conjugate or the pharmaceutical composition according to the invention may be administered to the human or animal subject in an amount or dosage that efficiently treats a disease or is sufficient for diagnostic purposes.

The antibody-linker conjugate or the pharmaceutical composition according to the invention may be administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The antibody-linker conjugate or the pharmaceutical composition according to the invention may be formulated, dosed, and administered in a fashion consistent of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody-linker conjugate or the pharmaceutical composition according to the invention need not be, but is optionally formulated with one or more agents currently used to prevent or treat the disorder in question. The effective amount of such other agents depends on the amount of antibody-linker conjugate present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of the antibody-linker conjugate or the pharmaceutical composition according to the invention (when used alone or in combination with one or more other additional therapeutic agents) will depend on the type of disease to be treated, the type of antibody-payload conjugate, the severity and course of the disease, whether the antibody-linker conjugate is administered for preventive or therapeutic purposes, previous therapy, the patient's clinical history and response to the antibody-linker conjugate, and the discretion of the attending physician. The antibody-linker conjugate or the pharmaceutical composition according to the invention is suitably administered to the patient at one time or over a series of treatments.

### EXAMPLES

### General Methods

The antibody Trastuzumab was commercially available (Herceptin®, Roche, bought from a pharmacy), as well as all the peptides-linkers and linker-payloads (custom synthesized by LifeTein and Levena Biopharma, respectively). Polatuzumab with heavy and light chain consisting of the sequences of SEQ ID NOs: 36 and 37 were transiently transfected into suspension-adapted CHO-K1 cells and expressed in serum-free/animal component-free media. The proteins were purified from the supernatants by Protein A affinity chromatography (Mab Select Sure column; GE Healthcare).

Conjugation reactions were performed by mixing of native, glycosylated monoclonal antibody, microbial transglutaminase (MTG, Zedira), and the indicated peptide-linker or linker-payload, in buffer in a rotating thermomixer. Conjugation efficiency was assessed by LC-MS under DTT reduced conditions. Reduction of samples was achieved by incubation of the samples for 15 min at 37° C. in 50 mM DTT (final) and 50 mM Tris buffer. Probes were analyzed on a Xevo G2-XS QTOF (Waters) coupled to an Acquity UPLC H-Class System (Waters) and an ACQUITY UPLC BEH C18 Column. Conjugation efficiency (CE) was calculated from deconvoluted spectra and presented in %. Intensities resulting from both glycoforms (G1F and G0F) were taken into account for the calculation, according to the formula:

$$CE \% = \frac{\sum ( {( Int ( G 0 F + G 1 F ) )}_{cj} )}{\sum {( Int ( G 0 F + G 1 F ) )}_{cj , ncj}}$$

With cj=conjugated and ncj=non-conjugated

### Example 1: Identification of Optimized Reaction Conditions

### Method

Reactions were performed using two different sets of reaction conditions: condition 1: 1 mg/ml of native, glycosylated Trastuzumab antibody, MTG at a concentration of 6 U/mg, and 80 molar equivalents of the indicated peptide-linker or linker-payload, in Tris 50 mM pH 7.6 for 20 hours, at 37° C. in a rotating thermomixer or condition 2: 5 mg/ml of native, glycosylated Trastuzumab antibody, MTG at a concentration of 5 U/mg, and 5 molar equivalents of the indicated peptide-linker or linker-payload, in Tris 50 mM pH 7.6 for 24 hours, at 37° C. in a rotating thermomixer. Conjugation efficiency was assessed by LCMS as described in general methods.

### Results

Surprisingly, excellent conjugation efficiencies were obtained using substantially less equivalents of peptide-linker and linker-payloads (80 molar equivalents vs. 5 molar equivalents) and a lower MTG (5 U/mg versus 6 U/mg) concentrations as shown in Table 3. Even more strikingly, conjugation efficiencies using functionalized peptides or linker-payloads were significantly increased using condition 2, i.e., using 5 molar of peptide-linker or linker-payload equivalents rather than 80. This observation was true for different linkers but also different payload classes (three cytotoxins: MMAE, Maytansin and Exatecan, as well as one steroid: cortisol), which is very surprising.

TABLE 3
Conjugation efficiencies using optimized reaction conditions

| | Conjugation | Conjugation |
|---|---|---|
| | efficiency | efficiency |
| | (%) with | (%) with |
| Peptide-linker or linker-payloads | conditions 1 | conditions 2 |
| RKAAR (SEQ ID NO: 43) | 92% | 95% |
| RKAAR-azide (SEQ ID NO: 43) | 59% | 97% |
| RKAAR-PABC-MMAE (SEQ ID NO: 43) | 5% | 99% |
| RKAAR-PABC-Exa (SEQ ID NO: 43) | 0% | 99% |
| RKAAR-EDA-CS (SEQ ID NO: 43) | 14% | 98% |
| RKAAR-May (SEQ ID NO: 43) | 5% | 100% |
| RRKAY (SEQ ID NO: 44) | 59% | 100% |
| RRKAY-azide (SEQ ID NO: 44) | 12% | 98% |
| RRKAY-PABC-MMAE (SEQ ID NO: 44) | 2% | 100% |
| RKAAR-May (SEQ ID NO: 44) | 0% | 89% |
| RRK | 51% | 99% |
| RRK-azide | 14% | 98% |
| RRK-PABC-MMAE | 0% | 96% |
| RRK-PABC-Exa | 0% | 83% |
| RRK-May | 0% | 87% |
| ARKRA (SEQ ID NO: 45) | 74% | 98% |
| ARKRA-azide (SEQ ID NO: 45) | 28% | 99% |
| ARKRA-PABC-MMAE (SEQ ID NO: 45) | 0% | 89% |
| ARKRA-May (SEQ ID NO: 45) | 0% | 87% |
| RKAA-PABC-MMAE (SEQ ID NO: 46) | 50% | 98% |
| KAR-PABC-MMAE | nt | 88% |
| May-C5-AAKR (SEQ ID NO: 47) | nt | 85% |
| HK | 42% | 90% |
| HK-PABC-Exa | 30% | 81% |
| AHK-PABC-Exa | 47% | 89% |
| HKA-PABC-Exa | 47% | 83% |

CS: cortisol,
May: maytansine,
Exa: exatecan,
nt: not tested

### Example 2: Identification of Further Reaction Conditions

To demonstrate that conjugation with lysine-linker-payloads tolerate a wide variety of the reaction conditions, linker-payload conjugation to Polatuzumab was performed using a range of reaction conditions with varying parameters.

### Method

As standard condition, the following parameters were used: 5 mg/ml of native, glycosylated polatuzumab antibody, MTG at a concentration of 5 U/mg, and 5 molar equivalents of RKAA-PABC-MMAE, in Tris 50 mM, pH 7.6, for 24 hours at 37° C. in a rotating thermomixer.

The variable parameters are shown in Table 4.

Conjugation efficiency was assessed by LCMS as follows: Conjugation efficiency (CE) was calculated from deconvoluted spectra and presented in %. Intensities resulting from both glycoforms (G1F and G0F) were taken into account for the calculation, according to the formula:

$$CE \% = \frac{\sum ( {( Int ( G 0 F + G 1 F ) )}_{cj} )}{\sum {( Int ( G 0 F + G 1 F ) )}_{cj , ncj}}$$

with cj=conjugated and ncj=non-conjugated

### Results

The RKAA-PABC-MMAE linker-payload conjugated with very high conjugation efficiency over a very large range of reaction conditions: Conjugation efficiencies of >80% were achieved using antibody concentration between 5 to 17 mg/ml, MTG concentration relative to antibody concentration (U/mg) between 2 and 10 U/mg. Further, high conjugation efficiencies were also obtained with molar concentrations of linker versus antibody (2 to 8 equivalents) as well as a very wide range of pH (from pH 6.0 with conjugation efficiency of 67% and pH 8 of 86%).

Surprisingly, higher conjugation efficiency was obtained with less linker-payload excess vs antibody, i.e., 2-20 equivalent of linker payload yielded higher conjugation efficiencies than using 80 equivalents, which is contrary to what can be expected. (Table 4).

TABLE 4
Conjugation efficiencies of RK-linker-payload
to Polatuzumab under different reaction conditions
Effects on reaction parameters on conjugation efficiency

| | Antibody final concentration (mg/mL) | | | | | |
|---|---|---|---|---|---|---|
| Parameters | 5 | 6 | 8 | 10 | 15 | 17 |
| CE (%) | 98% | 95% | 95% | 96% | 86% | 95% |

| | | MTG loading (U/mg) | | | | | | | |
|---|---|---|---|---|---|---|---|---|---|
| Parameters | | 2 | 3 | 3.5 | 4.5 | 5 | 6 | 9 | 10 |
| CE (%) | | 94% | 92% | 95% | 97% | 98% | 100% | 96% | 88% |
| Param- | Molar equivalent of RK-linker-payload vs antibody | | | | | | | | |
| eters | 2 | 2.5 | 3 | 4 | 5 | 6 | 8 | 20 | 80 |
| CE (%) | 70% | 81% | 80% | 92% | 98% | 94% | 93% | 71% | 50% |

| | pH | | | | | |
|---|---|---|---|---|---|---|
| Parameters | 6.0 | 6.5 | 7.0 | 7.5 | 7.6 | 8.0 |
| CE (%) | 67% | 86% | 91% | 90% | 98% | 86% |

### Example 3: Identification of Further Reaction Conditions

To demonstrate the importance of the linker concentration, further linkers with various sequences and payloads were conjugated to the antibodies Polatuzumab and Transtuzumab at varying concentrations.

### Method

As standard conditions, the following parameters were used: 3.5 mg/ml of native, glycosylated Polatuzumab or Trastuzumab antibody and MTG at a concentration of 5 U/mg, in Tris 50 mM, pH 7.6, for 24 hours at 37° C. in a rotating thermomixer. The linkers RKAA-PABC-MMAE, KAR-PABC-MMAE and AHK-PABC-Exa were added to the reaction mix at varying concentrations.

The variable parameters are shown in Table 5.

Conjugation efficiency was assessed by RPLC as follows: after reduction, samples were analyzed on a UHPLC Dionex UltiMate 3000 (Thermo Fisher) using BioResolve RP mAb Polyphenyl column. Conjugation efficiency (CE) was calculated using relative peak area extracted from the RPLC chromatogram according to the formula and presented in %:

$$CE \% = \frac{\sum ( {( Int ( relative peak area ) )}_{cj} )}{\sum {( Int ( relative peak area ) )}_{cj , ncj}}$$

With cj=conjugated and ncj=non-conjugated

### Results

With all tested linkers, exceptionally high conjugation efficiencies were obtained when 5-20 equivalents of linker payload were added to the reaction.

TABLE 5
Conjugation efficiencies of RKAA-PABC-MMAE,
KAR-PABC-MMAE, or AHK-PABC-Exa to Polatuzumab
or Trastuzumab under different reaction conditions
Effects on reaction parameters on conjugation efficiency

| | Molar equivalent of RKAA-PABC- | | | | | |
|---|---|---|---|---|---|---|
| | MMAE vs antibody (Polatuzumab) | | | | | |
| Parameters | 2 | 5 | 10 | 20 | 40 | 80 |
| CE (%) | 64% | 84% | 87% | 84% | 73% | 57% |
| | Molar equivalent of KAR-PABC- | | | | | |
| | MMAE vs antibody (Trastuzumab) | | | | | |
| Parameters | 2 | 5 | 10 | 20 | 40 | 80 |
| CE (%) | 63% | 84% | 82% | 93% | 87% | 75% |
| | Molar equivalent of AHK-PABC- | | | | | |
| | Exa vs antibody (Trastuzumab) | | | | | |
| Parameters | 2 | 5 | 10 | 20 | 40 | 80 |
| CE (%) | 76% | 91% | 93% | 89% | 72% | 47% |

1. A method for producing an antibody-linker conjugate by means of a transglutaminase, the method comprising a step of conjugating a linker comprising the structure (shown in N→C direction)
(Sp₁)-K-(Sp₂)-B-(Sp₃) or(Sp₁)-B-(Sp₂)-K-(Sp₃)
to a Gln residue comprised in an antibody, wherein
(Sp₁) is a chemical spacer or is absent;
(Sp₂) is a chemical spacer or is absent;
(Sp₃) is a chemical spacer or is absent;
K is lysine or a lysine derivative or a lysine mimetic;
B is a linking moiety or a payload;
wherein the linker is conjugated to the Gln residue comprised in the antibody via a primary amine comprised in the side chain of the lysine residue, the lysine derivative or the lysine mimetic; and
wherein the antibody is contacted with less than 80 molar equivalents of the linker.

2. The method according to claim 1, wherein the Gln residue to which the linker is conjugated is Gln residue Q295 (EU numbering) of the C_{H}2 domain of an IgG antibody.

3. The method according to claim 2, wherein the IgG antibody is a glycosylated IgG antibody, optionally wherein the IgG antibody is glycosylated at residue N297 (EU numbering) of the C_{H}2 domain.

4. The method according to claim 1, wherein the antibody is contacted with 20 molar equivalents or less than 20 molar equivalents of the linker, optionally wherein the antibody is contacted with 2-20 molar equivalents of the linker.

5. (canceled)

6. The method according to claim 1, wherein the antibody is added to the conjugation reaction at a concentration ranging from 1-50 mg/mL.

7. The method according to claim 1, wherein the transglutaminase is added to the conjugation reaction at a concentration ranging from 1-20 U/mg antibody.

8. The method according to claim 1, wherein conjugation of the linker to the antibody is achieved at a pH ranging from 6 to 8.5.

9. The method according to claim 1, wherein the chemical spacers (Sp₁), (Sp₂) and (Sp₃) each independently comprise between 0 and 12 amino acid residues.

10. The method according to claim 1, wherein
(a) the linker comprises not more than 25, 20, 15, 14, 13, 12, 11, 10, 9, 8, 7, 6, 5, 4 amino acid residues,
(b) the net charge of the linker is neutral or positive; and/or
(c) the linker comprises no negatively-charged amino acid residues.

11. (canceled)

12. (canceled)

13. The method according to claim 1, wherein B is a linking moiety.

14. The method according to claim 13, wherein the linking moiety B comprises
a bioorthogonal marker group, or
a non-bio-orthogonal entity for crosslinking, optionally wherein
the bioorthogonal marker group or the non-bio-orthogonal entity for crosslinking consists of or comprises at least one molecule or moiety selected from the group consisting of:
—N—N≡N, or —N₃;
Lys(N₃);
a tetrazine;
an alkyne;
a strained cyclooctyne;
BCN;
a strained alkene;
a photoreactive group;
an aldehyde;
an acyltrifluoroborate;
a protein degradation agent (‘PROTAC’);
a cyclopentadiene/spirolocyclopentadiene;
a thio-selective electrophile;
—SH; and
cysteine.

15. (canceled)

16. The method according to claim 13, the method comprising a further step of conjugating one or more payloads to the linking moiety B, optionally wherein the one or more payloads is conjugated to the linking moiety B via a click-reaction.

17. (canceled)

18. The method according to claim 1, wherein B is a payload.

19. The method according to claim 18, wherein the payload comprises at least one of:
a toxin, optionally wherein the toxin is selected from the group consisting of a pyrrolobenzodiazepine, an auristatin, a maytansinoid, a duocarmycin, a nicotinamide phosphoribosyltransferase (NAMPT) inhibitor, a tubulysin, an enediyne, an anthracycline derivative (PNU), a pyrrole-based kinesin spindle protein (KSP) inhibitor, a cryptophycin, a drug efflux pump inhibitor, a sandramycin, an amanitin, and a camptothecin;
a cytokine;
a growth factor;
a radionuclide;
a hormone;
an anti-viral agent;
an anti-bacterial agent;
a fluorescent dye;
an immunoregulatory/immunostimulatory agent;
a half-life increasing moiety;
a solubility increasing moiety;
a polymer-toxin conjugate;
a nucleic acid;
a biotin or streptavidin moiety;
a vitamin;
a protein degradation agent (‘PROTAC’);
a target binding moiety; and/or
an anti-inflammatory agent.

20. (canceled)

21. The method according to claim 1, wherein the chemical spacer (Sp₂) comprises a self-immolative moiety, optionally wherein the self-immolative moiety is directly attached to the payload B, optionally wherein the self-immolative moiety comprises a p-aminobenzyl carbamoyl (PABC) moiety or a self-immolative amino methylene spacer.

22. (canceled)

23. (canceled)

24. The method according to claim 1, wherein the antibody is an IgG antibody, optionally wherein the antibody is selected from the group consisting of: Brentuximab, Trastuzumab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Sacituzumab, Belantamab, Polatuzumab and Enfortumab.

25. (canceled)

26. The method according to claim 1, wherein the linker is conjugated to the γ-carboxamide group of the Gln residue comprised in the antibody, optionally wherein the linker is suitable for conjugation to a glycosylated antibody with a conjugation efficiency of at least 20%, 30%, 40%, 50%, 60%, 70%, 75%, 80%, 85%, 90% or 95%.

27. (canceled)

28. The method according to claim 1, wherein the transglutaminase is a microbial transglutaminase, optionally derived from a Streptomyces species, optionally wherein the Streptomyces species is Streptomyces mobaraensis.

29. An antibody-linker conjugate which has been produced with a method according to claim 1.

30-32. (canceled)

33. A method of treating a patient
suffering from,
being at risk of developing, and/or
being diagnosed for
a neoplastic disease, a neurological disease, an autoimmune disease, an inflammatory disease or an infectious disease, the method comprising administering the antibody-linker conjugate of claim 29 to the subject, wherein the antibody-linker conjugate comprises at least one toxin.

34-37. (canceled)
